(19) United States
(12) Patent Application Publication
(10) Pub. No.: US 2006/0121448 A1
(43) Pub. Date: Jun. 8, 2006

(54) Method for inducing complete hepatitis c virus (hcv) replication in vitro

(75) Inventors: Nahum Sonenberg, Quebec (CA); Marcelo Lopez-Lastra, Santiago (CL)

Correspondence Address: FULBRIGHT & JAWORSKI, LLP, 600 CONGRESS AVENUE, SUITE 2400, AUSTIN, TX, 78701, US

(73) Assignee: McGill University, Montreal (CA)

(21) Appl. No.: 10/523,602

(22) PCT Filed: Aug. 6, 2003
(86) PCT No.: PCT/CA03/01184

§ 371 Date: Sep. 21, 2005

Related U.S. Application Data
(60) Provisional application No. 60/401,005, filed on Aug. 6, 2002.

(51) Int. Cl. C12Q 1/70; C12N 15/86; C12N 7/00

(52) U.S. Cl. 435/5; 435/235.1; 435/325; 435/456

(57) ABSTRACT

The present invention relates to hepatitis C virus (HCV). More particularly, the invention relates to the development of a tool suitable for the search, discovery and validation of novel HCV antiviral drugs and therapies (e.g. vaccine). The invention further relates to methods for inducing HCV replication in vitro, and more particularly to a simple in vitro replication assay of HCV which enables productive and sustained infectious HCV production.

### FIELD OF THE INVENTION

The present invention relates to hepatitis C virus (HCV). More particularly, the invention relates to the development of a tool suitable for the search, discovery and validation of novel HCV antiviral drugs and therapies (e.g. vaccine). The invention further relates to methods for inducing HCV replication in vitro, and more particularly to a simple in vitro replication assay for HCV. In addition, the invention relates to the use of the methods of the present invention to prognose the resistance/sensitivity of a particular strain of HCV to a chosen anti-HCV agent. In one embodiment, the present invention relates to an adaptation of a therapeutic regimen for a patient infected with HCV which takes into account the resistance/sensitivity phenotype of the HCV strain which infects same.

### BACKGROUND OF THE INVENTION

The hepatitis C virus (HCV) is an enveloped RNA virus of the Flaviviridae, which is classified within the Hepacivirus genus. HCV is an important etiologic agent of chronic liver diseases. At this time HCV infection is one of the primary causes of liver transplantation in the US and other countries. Acute infections are usually subclinical or associated with mild symptoms, but the virus persists in more than 80% of infected individuals despite evidence of active, antiviral immunological response (Hepatol 1998, 28:939-944). It is estimated than more than 170,000,000 people are seropositive world-wide (Hepatology 1997, 26:62S-6S). The long-term outcome of HCV persistent infections are varied, and they can range from an apparently, healthy carrier state to chronic active hepatitis, liver cirrhosis, and eventually hepatocellular carcinoma (N Engl J Med 1992, 327:1899-1905). The mechanism of such pervasive persistence is unknown. To date, there is no vaccine for HCV and the most effective therapy is treatment with peginterferon in combination with the nucleoside analogue ribavirin (Clin Liver Dis 7, 149-61 (2003), Nat Rev Drug Discov 1, 867-81 (2002). Unfortunately, during IFN-α treatment selection of viral variants resistant to INF-α occurs frequently (Microbes & Infection 200, 2:1743-1756). In addition, ribavirin can be used to treat patients. HCV resistance to ribavirin is also common. The search for HCV drugs as well as the development of an HCV vaccine is severely hampered by the lack of an efficient tissue culture or simple animal system for the study of replication and HCV pathogenicity. The only animal models currently available for the study of this virus are the chimpanzee and a mouse which possesses a chimeric human liver (Antiviral Research 2001, 52:1-17; Nat Med 2001, 7:927-933). These facts cast HCV as an emerging human pathogen of extreme medical significance (J Viral Hepat 1999, 6:35-47).

There thus remains a need to provide a simple assay for HCV replication which would enable the study of HCV replication and/or pathogenesis and enable the development of a treatment or prophylaxis for HCV infections. There also remains a need to provide a HCV replication system which enables the screening, discovery and validation of novel anti-HCV compounds which can act in a larger number of stages of the HCV life cycle such as entry, replication, translation, assembly, trafficking and release. There also remains a need to provide a system which enables the replication of HCV from a patient so as to enable simpler and more efficient genotyping thereof and/or phenotyping (e.g. to identify its resistance/sensitivity characteristics toward anti-viral compounds). Virologics patent and patent applications no. US20030008282A1 published Jan. 9, 2003, U.S. Pat. No. 6,242,187 issued Jun. 5, 2001 and U.S. Pat. No. 5,837,464 issued Nov. 17, 1998 describe methods for determining anti-viral drug susceptibility and resistance.

While HCV infects a large number of individuals, no efficient treatment or vaccine has been developed, despite a significant effort by the pharmaceutical industry. Thus, most companies with existing programs in the anti-infective area are focused towards the discovery of agents that are active against this virus. Until now,; the human immunodeficiency virus (HIV) has provided a useful strategy for HCV antiviral drug development (Drug Discov. Today 1999, 4:518-529). In fact, the understanding of the function of anti-HIV drugs has outlined the research platform of most of the companies screening for anti-HCV drugs. Both viruses share interesting features. They lead to chronic infection, are highly mutable, and they code for specific enzymes that are not expected to be present in a normal non-infected cell. Based on the results of HIV therapy, it is likely that a combination therapy involving at least two drugs directed against separate targets will be more effective at reducing HCV load (quantity of virus in the serum), and minimizing the emergence of resistant strains than monotherapy. As the selected targets against HIV have been the viral encoded protease and the viral reverse transcriptase, it is not surprising to find that HCV protease and RNA dependent RNA polymerase have often been mentioned as candidate antiviral targets. As judged by the lack of disclosures, the discovery of anti-HCV agents has not been successful despite the functional similarity of several HCV-enzymes with known targets from other antiviral programs. Admittedly, part of this failure is because of the lack of a tissue culture system, which in turn limits primary screens to isolate viral protein targets. Interestingly, despite the fact that the enzyme assays to test HCV protease are known, the discovery of a potential drug candidate has met with little success. Taken together, it might be concluded that putative chemotypes for inhibition of HCV-targets are poorly represented in most industrial compound collections (Drug Discov. Today, 1999, 4:518-529).

Should a series of novel anti-HCV drugs be developed, to advance these agents into the drug development pipeline, several issues will need to be addressed, notably, their mechanism of action. Unfortunately, tissue culture and in vivo control experiments using whole virus are required to better determine the mode of inhibition. As stated above, an efficient cell culture system for the replication of HCV has not yet been provided (Drug Discov Today 1999, 4:518-529; Antiviral Res. 2001, 52:1-17; J. Mol. Biol. 2001, 313:451-464; Virus Res. 2002, 82:35-32).

Attempts have been made to provide an in vitro culture system for HCV, based on the use of human cells of hepatocytic and lymphocytic origin, but low and variable levels of replication and virus-induced cytotoxicity posed important problems. Primary hepatocytes (derived from a human donor) can be infected with HCV isolated from serum of infected patients, and the virus can be detected in the supematant for several weeks after infection. HCV replication has been demonstrated by detection of minus-strand RNA, an intermediate of virus replication, in primary hepatocytes derived from a HCV-negative donor after infection with sera from HCV-positive patients. However, the availability of primary hepatocytes is limited. In addition, their isolation is time-consuming and labor-intensive. Consequently, such tissue culture systems are generally considered unsuitable for intensive large-scale antiviral studies.

Another example of progress in this domain has been the construction of subgenomic selective replicons cloned from a full-length HCV consensus genome from an infected liver (Antiviral Res. 2001, 52:1-17; J. Mol. Biol. 2001, 313:451-64; Virus Res. 2002, 82:25-32). Following transfection in human hepatoma cells, these RNAs were found to replicate to high levels, allowing detailed molecular studies of HCV and testing of antiviral drugs. One drawback of this system, however, is that it only expresses the non-structural viral proteins (Science 1999, 285:110-3). Therefore, studies aimed at assessing target viral assembly and trafficking through the cytoplasm cannot be carried out, with this reconstituted viral system. In other words, such artificial system is of a more limited potential to identify antiviral agents.

As previously mentioned animal models currently exist to study HCV replication. Although the chimpanzee model has contributed significantly to the understanding of HCV infection, the high cost and availability of these animals limit the extent to which antiviral-drug or therapy studies can be carried out. Small laboratory animals, including mice, are not susceptible to infection with HCV. An alternative model such as a mouse model with a chimeric human liver has been generated (Nat Med 2001, 7:927-933). This system is considered laborious and is known to require special expertise to isolate and transplant human hepatocytes and maintain a colony of fragile immunodeficient mice with an approximately 35% mortality in newborns due to a defect in blood coagulation (Nature Med. 2001, 7:927-933). Nevertheless, when all the required conditions are met this mouse model can provide an interesting system for testing antiviral agents.

There thus remains a need to provide a simple in vitro system, which is suitable for the replication of HCV.

There also remains a need to provide an in vitro tissue culture system for the complete replication of HCV.

There further remains a need to provide a tissue culture system for HCV which enables the screening, discovery, validation and further development of drugs and therapies for essentially all the different stages of virus replication such as virus entry, replication [viral (−) and (+) strand synthesis], viral protein synthesis, virus assembly, virus trafficking, and virus release.

Also, there remains a need to provide a suitable cellular system which enables a quick enough assessment of the phenotype and/or genotype of one or more HCV infecting a patient, to adapt or improve the treatment thereof.

The present invention seeks to meet these and other needs.

The present description refers to a number of documents, the content of which is herein incorporated by reference in their entirety.

### SUMMARY OF THE INVENTION

The invention relates to a simple in vitro culture system, which is suitable for the full replication cycle of hepatitis C virus (HCV).

The invention further relates to an in vitro culture system, which is suitable for the replication of hepatitis C virus (HCV), comprising: HCV-infected cells cultivated in the presence of an HCV-activating composition, said activating composition comprising at least one mitogen; and a non-infected cell type which is infectable with HCV, whereby said activating composition enables a full replication cycle of said HCV in both the originally infected cells and non-infected cell type. In specific embodiments, the activating composition also comprises a cytokine. In more specific embodiments, the activating composition is selected from the group consisting of a) phytohaemagglutinin and IL-2; b) Staphylococcus aureus crown I (SAC) and IL-4; and c) SAC, IL2 and IL-4.

In addition, the invention relates to a tissue culture system for HCV which enables the screening, discovery, validation and further development of drugs and therapies for essentially all the different stages of virus replication such as virus entry, replication [viral (−) and (+) strand synthesis], viral protein synthesis, virus assembly, virus trafficking, and virus release.

The present invention also provides the means to diagnose HCV. In addition, it enables an identification of the response of a particular strain of HCV, from a particular patient, to a candidate antiviral compound or to a known antiviral compound.

In a particular embodiment, the invention provides the means to assess for sensitivity or resistance of a particular HCV strain to a known antiviral compound or candidate antiviral compound. In a related embodiment, such assessment enables an adaptation of the therapeutic regimen to better suit the sensitivity profile of the particular HCV strain.

In a specific embodiment, there is provided an assay for screening a test agent and selecting an agent which possesses anti-HCV activity, comprising: a) growing a HCV infected cell according to an in vitro assay of the present invention; and b) assaying replication, translation, assembly infection or the like of HCV.

In an other particular embodiment, the invention provides a method for identifying, from a library of compounds, a compound with anti-HCV activity, comprising: a) providing a screening assay comprising a measurable biological activity of HCV; b) contacting said screening assay with a test compound; and c) detecting if said test compound inhibits the biological activity of HCV; wherein a test compound which inhibits said biological activity is a compound with said inhibitory effect. In a specific embodiment of such method, the test compound with the therapeutic effect is further modified by combinatorial or medicinal chemistry to provide further analogs of the test compound also having the therapeutic effect.

In an other particular embodiment, the invention provides a compound having therapeutic effect on HCV, comprising: a) providing a screening assay comprising a measurable biological activity of HCV; b) contacting the screening assay with a test compound; and c) detecting if the test compound inhibits the biological activity of HCV, wherein a test compound which inhibits said biological activity is a compound with said inhibitory effect. In a specific embodiment, the compound with the therapeutic effect is further modified by combinatorial or medicinal chemistry to provide analogs of the compound also having said therapeutic effect.

In another embodiment, the invention enables the phenotyping and/or genotyping of a particular HCV strain.

The present invention further relates to a method of activating the replication of HCV in peripheral blood mononuclear cells (PBMCs) comprising obtention of same from a HCV-infection patient and activating the replication of HCV by incubating the PBMCs with an activation-inducing amount of at least one mitogen (e.g. activator).

The invention in addition relates to a co-culturing system for replicating HCV in vitro which comprises co-culturing PBMCs (or peripheral blood lymphocytes (PBLs)) infected with HCV, wherein the PBMCs have been activated and in which the HCV can actively replicate, together with a cell line, wherein the co-culturing enables infection of a naive cell line and replication of the HCV thereinto. In a particular embodiment of the present invention, the cell line is an immortalized cell line.

The invention in addition relates to a method of generating a vaccine to HCV comprising a pulsing of monocyte-derived dendritic cells (DCs) with HCV, co-cultured with autologous peripheral blood lymphocytes from a HCV-seropositive individual. In a more specific embodiment, the method further comprises a selection of clonal T cell populations that are responsive to the virus and an injection of these HCV responsive T-cell populations to the original donor.

It is believed that the Applicant is the first to provide an in vitro cell system which enables replication of a native HCV.

It is believed that prior to the present invention, while HCV was known to infect PBMCs it was unknown that it could actively replicate in them. Thus, the present invention demonstrates HCV tropism for PBMCs and more particularly for PBLCs. As known in the art, PBMCs are a mixture of cells which also include macrophages and PBLCs (which can be obtained from PBMCs and contain about T cells, about 85%) and B cells, about 5% as estimated from non-infected patents).

It is also believed that this is the first demonstration that the HCV produced in an in vitro system is infectious and that sustainable replication of HCV can be achieved.

Before the present invention, large-scale production of HCV was unthinkable. The methods and in vitro system of the present invention enables active replication of HCV in primary cells for 7 to 9 days depending on the host cells and opens the way to large scale production.

Prior to the present invention, no tissue culture technology currently existed to replicate HCV. The only animal models currently available for the study of this virus are the chimpanzee and mice models (mice with chimeric human livers). These animal based-systems are laborious and require special expertise and facilities.

In order to provide a clear and consistent understanding of terms used in the present description, a number of definitions are provided hereinbelow.

Nucleotide sequences are presented herein by single strand, in the 5′ to 3′ direction, from left to right, using the one letter nucleotide symbols as commonly used in the art and in accordance with the recommendations of the IUPAC-IUB Biochemical Nomenclature Commission.

Unless defined otherwise, the scientific and technological terms and nomenclature used herein have the same meaning as commonly understood by a person of ordinary skill to which this invention pertains. Generally, the procedures for cell cultures, Infection, molecular biology methods and the like are common methods used in the art: Such standard techniques can be found in reference manuals such as for example Sambrook et al. (1989, Molecular Cloning—A Laboratory Manual, Cold Spring Harbor Laboratories) and Ausubel et al. (1994, Current Protocols in Molecular Biology, Wiley, New York).

The present description refers to a number of routinely used recombinant DNA (rDNA) technology terms. Nevertheless, definitions of selected examples of such rDNA terms are provided for clarity and consistency.

As used herein the terms ‘activator’ and ‘inducer’ refer to molecules which can trigger HCV replication in the culture system of the present invention. Inducement of HCV replication in the patient's infected cells require activation. This activation can be effected by a number of molecules. Non-limiting examples of mitogens which can be used as activators include receptor mediated activators and receptor independent activator such as: for T-cells: phytohaemagglutinin (PHA), concanavalin A, pokeweed, phorbolester, anti-CD3, superantigens, antigens that are presented by APC; for B-Cells: SAC, Staphilococcal protein A, CD40 ligand, antiimmunoglobulins, bacterial lipopolysaccharides (LPS). Cytokines such as for example IL2, IL-4. IL5, IL6, IL10, IL13 can also be used to further induce HCV replication. In one embodiment, there is used a mixture of activators such as PHA and IL-2; SAC and IL-4, SAC and IL2 and IL-4. In order to activate the infected cell, at least one mitogen can be used. A cocktail of at least one mitogen with at least one cytokine was shown to trigger significant activation of HCV replication. IFN could also be used.

As used herein, “nucleic acid molecule”, refers to a polymer of nucleotides. Non-limiting examples thereof include DNA (e.g. genomic DNA, cDNA), RNA molecules (e.g. mRNA) and chimeras thereof. The nucleic acid molecule can be obtained by cloning techniques or synthesized. DNA can be double-stranded or single-stranded (coding strand or non-coding strand [antisense, RNA]). RNA interference (RNAI) can be used in accordance with the present invention using, for example, the teachings of U.S. Pat. No. 6,506,559.

The term “recombinant DNA” as known in the art refers to a DNA molecule resulting from the joining of DNA segments. This is often referred to as genetic engineering. The same is true for “recombinant nucleic acid”.

The term “DNA segment, is used herein, to refer to a DNA molecule comprising a linear stretch or sequence of nucleotides. This sequence when read in accordance with the genetic code, can encode a linear stretch or sequence of amino acids which can be referred to as a polypeptide, protein, protein fragment and the like.

The terminology “amplification pair” refers herein to a pair of oligonucleotides (oligos) of the present invention, which are selected to be used together in amplifying a selected nucleic acid sequence by one of a number of types of amplificaton processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

The nucleic acid (e.g. DNA or RNA) for practicing the present invention may be obtained according to well known methods.

Oligonucleotide probes or primers of the present invention may be of any suitable length, depending on the particular assay format and the particular needs and targeted genomes employed. In general, the oligonucleotide probes or primers are at least 12 nucleotides in length, preferably between 15 and 24 molecules, and they may be adapted to be especially suited to a chosen nucleic acid amplification system. As commonly known in the art, the oligonucleotide probes and primers can be designed by taking into consideration the melting point of hybridization thereof with its targeted sequence (see below and in Sambrook et al., 1989, Molecular Cloning—A Laboratory Manual, 2nd Edition, CSH Laboratories; Ausubel et al., 1989, in Current Protocols in Molecular Biology, John Wiley & Sons Inc., N.Y.).

The term “DNA” molecule or sequence (as well as sometimes the term “oligonucleotide”) refers to a molecule comprised generally of the deoxyribonucleotides adenine (A), guanine (G), thymine (T) and/or cytosine (C), often in a double-stranded form, and comprises or includes a “regulatory element” according to the present invention, as the term is defined herein. The term “oligonucleotide” or “DNA” can be found in linear DNA molecules or fragments, viruses, plasmids, vectors, chromosomes or synthetically derived DNA.

“Nucleic acid hybridization” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 1989, supra and Ausubel et al., 1989, supra) and are commonly known in the art. In the case of a hybridization to a nitrocellulose filter, as for example in the well known Southern blotting procedure, a nitrocellulose filter can be incubated overnight at 65° C. with a labeled probe in a solution containing 50% formamide, high salt (5×SSC or 5×SSPE), 5×Denhardt's solution, 1% SDS, and 100 μg/ml denatured carrier DNA (e.g. salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). The selected temperature is based on the melting temperature (Tm) of the DNA hybrid. Of course, RNA-DNA hybrids can also be formed and detected. In such cases, the conditions of hybridization and washing can be adapted according to well-known methods by the person of ordinary skill. Stringent conditions will be preferably used (Sambrook et al., 1989, supra).

Probes of the invention can be utilized with naturally occurring sugar-phosphate backbones as well as modified backbones including phosphorothioates, dithionates, alkyl phosphonates and α-nucleotides and the like. Modified sugar-phosphate backbones are generally taught by Miller, 1988, Ann. Reports Med. Chem. 23:295 and Moran et al., 1987, Nucleic Acids Res., 14:5019. Probes of the Invention can- be constructed of either ribonucleic acid (RNA) or deoxyribonucleic acid (DNA), and preferably of DNA.

The types of detection methods in which probes can be used include Southern blots (DNA detection), dot or slot blots (DNA, RNA), and Northern blots (RNA detection). Labeled proteins could also be used to detect a particular nucleic acid sequence to which it binds. Other detection methods include kits containing probes on a dipstick setup and the like.

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods (Sambrook et al., 1989, supra). Non-limiting examples of labels include ³H, ¹⁴C, ³²P, and ³⁵S. Non-limiting examples of detectable markers include ligands, fluorophores, chemiluminescent agents, enzymes, and antibodies. Other detectable markers for use with probes, which can enable an increase in sensitivity of the method of the invention, include biotin and radionudeotides. It will become evident to the person of ordinary skill that the choice of a particular label dictates the manner in which it is bound to the probe.

As commonly known, radioactive nucleotides can be incorporated into probes of the invention by several methods. Non-limiting examples thereof include kinasing the 5′ ends of the probes using gamma ³²P ATP and polynucleotide kinase, using the Klenow fragment of Pol I of E. coli in the presence of radioactive dNTP (e.g. uniformly labeled DNA probe using random oligonucleotide primers in low-melt gels), using the SP6/T7 system to transcribe a DNA segment in the presence of one or more radioactive NTP, and the like.

As used herein, “oligonucleotides” or “oligos” define a molecule having two or more nucleotides (ribo or deoxyribonudeotides). The size of the oligo will be dictated by the particular situation and ultimately on the particular use thereof and adapted accordingly by the person of ordinary skill. An oligonucleotide can be synthesized chemically or derived by cloning according to well known methods. While they are usually in a single-stranded form, they can be in a double-stranded form and even contain a “regulatory region”.

As used herein, a “primer” defines an oligonucleotide which is capable of annealing to a target sequence, thereby creating a double stranded region which can serve as an initiation point for DNA synthesis under suitable conditions. Primers can be, for example, designed to be specific for certain strains of HCV or chosen regions of HCV genome. In accordance with one embodiment of the present invention, the use of an “allele” or strain-specific primer with the other necessary reagents would give rise to an amplification product only when the “allele” or strain-specific sequence associated with a particular phenotype is present in the sample. The “wild type” allele would not give rise to an amplicon.

Amplification of a selected, or target, nucleic acid. sequence may be carried out by a number of suitable methods. See generally Kwoh et al., 1990, Am. Biotechnol. Lab. 8:14-25. Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the Qβ replicase system and NASBA (Kwoh et al., 1989, Proc. Natl. Acad. Sci. USA 86, 1173-1177; Lizardi et al., 1988, BioTechnology 6:1197-1202; Malek et al., 1994, Methods Mol. Biol., 28:253-260; and Sambrook et al., 1989, supra). Preferably, amplification will be carried out using PCR.

Polymerase chain reaction (PCR) is carried out In accordance with known techniques. See, e.g., U.S. Pat. Nos. 4,683,195; 4,683,202; 4,800,159; and 4,965,188 (the disclosures of all three U.S. patent are incorporated herein by reference). In general, PCR involves, a treatment of a nucleic acid sample (e.g., in the presence of a heat stable DNA polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analyzed to assess whether the sequence or sequences to be detected are present. Detection of the amplified sequence may be carried out by visualization following EtBr staining of the DNA following gel electrophores, or using a detectable label in accordance with known techniques, and the like. For a review on. PCR techniques (see PCR Protocols, A Guide to Methods and Amplifications, Michael et al. Eds, Acad. Press, 1990).

Ligase chain reaction (LCR) is carried out in accordance with known techniques (Weiss, 1991, Science 254:1292). Adaptation of the protocol to meet the desired needs can be carried out by a person of ordinary skill. Strand displacement amplification (SDA) is also carried out in accordance with known techniques or adaptations thereof to meet the particular needs (Walker et al., 1992, Proc. Natl. Acad. Sci. USA 89:392-396; and ibid., 1992, Nucleic Acids Res. 20:1691-1696).

As used herein, the term “gene” is well known in the art and relates to a nucleic acid sequence which usually defines a single protein or polypeptide. In this context, a “structural gene” defines a DNA sequence which is transcribed into RNA and translated into a protein having a specific amino add sequence thereby giving rise to a specific polypeptide or protein. It will be readily recognized by the person of ordinary skill, that the nucleic acid sequences of the present invention can be incorporated into anyone of numerous established kit formats which are well known in the art. It should be understood that in view of the occurrence of alternative splicing or other mRNA editing processes, or protein editing, more than one protein or polypeptide can be encoded from one gene. Thus, the term “gene”, as used herein, should not be limited to genes which only encode one protein.

A “heterologous” (e.g. a heterologous gene) region of a DNA molecule is a subsegment of DNA within a larger segment that is not found in association therewith in nature. The term “heterologous” can be similarly used to define two polypeptidic segments not joined together in nature. Non-limiting examples of heterologous genes include reporter genes such as luciferase, chloramphenicol acetyl transferase, β-galactosidase, and the like which can be juxtaposed or joined to heterologous control regions or to heterologous polypeptides.

The term “vector” is commonly known in the art and defines a plasmid DNA, phage DNA, viral DNA and the like, which can serve as a DNA vehicle into which DNA of the present invention can be cloned. Numerous types of vectors exist and are well known in the art.

The term “expression” defines the process by which a gene is transcribed into mRNA (transcription), the mRNA is then being translated (translation) into one polypeptide (or protein) or more.

The terminology “expression vector defines a vector or vehicle as described above but designed to enable the expression of an inserted sequence following transformation into a host.

The cloned gene (inserted sequence) is usually placed under the control of control element sequences such as promoter sequences. The placing of a cloned gene under such control sequences is often referred to as being operably linked to control elements or sequences.

Operably linked sequences may also include two segments that are transcribed onto the same RNA transcript Thus, two sequences, such as a promoter and a “reporter sequence” are operably linked if transcription commencing in the promoter will produce an RNA transcript of the reporter sequence. In order to be “operably linked” it is not necessary that two sequences be immediately adjacent to one another.

Expression control sequences will vary depending on whether the vector is designed to express the operably linked gene in a prokaryotic or eukaryotic host or both (shuttle vectors) and can additionally contain transcriptional elements such as enhancer elements, termination sequences, tissue-specificity elements, and/or translational initiation and termination sites.

Prokaryotic expressions are useful for the preparation of large quantities of the protein encoded by the DNA sequence of interest. This protein can be purified according to standard protocols that take advantage of the intrinsic properties thereof, such as size and charge (e.g. SDS gel electrophloresis, gel filtration, centrifugation, ion exchange chromatography). In addition, the protein of interest can be purified via affinity chromatography using polyclonal or monoclonal antibodies. The purified protein can be used for therapeutic applications.

The DNA construct can be a vector comprising a promoter that is operably linked to an oligonucleotide sequence of the present invention, which is in turn, operably linked to a heterologous gene, such as the gene for the luciferase reporter molecule. “Promoter” refers to a DNA regulatory region capable of binding directly or indirectly to RNA polymerase in a cell and initiating transcription of a downstream (3′ direction) coding sequence. For purposes of the present invention, the promoter is preferably bound at its 3′ terminus by the transcription initiation site and extends upstream (5′ direction) to Include the minimum number of bases or elements necessary to initiate transcription at levels detectable above background. Within the promoter will be found a transcription initiation site (conveniently defined by mapping with S1 nuclease), as well as protein binding domains (consensus sequences) responsible for the binding of RNA polymerase. Eukaryotic promoters will often, but not always, contain “TATA” boxes and “CCAT” boxes. Prokaryotic promoters contain -10 and -35 consensus sequences, which serve to initiate transcription and the transcript products contain Shine-Dalgarno sequences, which serve as ribosome binding sequences during translation initiation.

As used herein, the designation “functional derivative”, denotes, in the context of a functional derivative of a sequence whether a nucleic acid or amino acid sequence, a molecule that retains a biological activity (either functional or structural) that is substantially similar to that of the original sequence. This functional derivative or equivalent may be a natural derivative or may be prepared synthetically. Such derivatives include amino acid sequences having substitutions, deletions, or additions of one or more amino acids, provided that the biological activity of the protein is conserved. The same applies to derivatives of nucleic acid sequences which can have substitutions, deletions, or additions of one or more nucleotides, provided that the biological activity of the sequence is generally maintained. When relating to a protein sequence, the substituting amino acid generally has chemico-physical properties which are similar to that of the substituted amino acid. The similar chemico-physical properties include, similarities in charge, bulkiness, hydrophobicity, hydrophylicity and the like. The term “functional derivatives” is intended to include “fragments”, segments”, “variants”, “analogs' or “chemical derivatives” of the subject matter of the present invention.

It should be understood that some variants of protein or nucleic acid molecule of the invention might have substantially dissimilar biological interaction with a particular compound as compared to a “wild type” counterpart. For example, a particular mutation might render the HCV strain resistant to a particular compound or group of compounds.

The functional derivatives of the present invention can be synthesized chemically or produced through recombinant DNA technology. All these methods are well known in the art.

As used herein, “chemical derivatives” is meant to cover additional chemical moieties not normally part of the subject matter of the invention. Such moieties could affect the physico-chemical characteristic of the derivative (e.g. solubility, absorption, half life, decrease of toxicity and the like). Such moieties are exemplified in

Remington's Pharmaceutical Sciences (1980). Methods of coupling these chemical-physical moieties to a polypeptide or nucleic acid sequence are well known in the art.

The term “allele” defines an alternative form of a gene.

As commonly known, a “mutation” is a detectable change in the genetic material which can be transmitted to a daughter cell. As well known, a mutation can be, for example, a detectable change in one or more deoxyribonucleotide. For example, nucleotides can be added, deleted, substituted for, inverted, or transposed to a new position. Spontaneous mutations and experimentally induced mutations exist. A mutant polypeptide can be encoded from this mutant nucleic acid molecule.

As used herein, the term “purified” refers to a molecule having been separated from a cellular component. Thus, for example, a “purified protein” has been purified to a level not found in nature. A “substantially pure” molecule is a molecule that is lacking in most other cellular components.

As used herein, the terms “molecule”, “compound”, “agent” or “ligand” are used interchangeably and broadly to refer to natural, synthetic or semi-synthetic molecules or compounds. The term “molecule” therefore denotes for example chemicals, macromolecules, cell or tissue extracts (from plants or animals) and the like. Non-limiting examples of molecules indude nucleic acid molecules, peptides, antibodies, carbohydrates and pharmaceutical agents. The agents can be selected and screened by a variety of means including random screening, rational selection and by rational design using for example protein or ligand modeling methods such as computer modeling. The terms “rationally selected” or “rationally designed” are meant to define compounds which have been chosen based on the configuration of interacting domains of the present invention. As will be understood by the person of ordinary skill, macromolecules having non-naturally occurring modifications are also within the scope of the term “molecule”. For example, peptidomimetics, well known in the pharmaceutical industry and generally referred to as peptide analogs can be generated by modeling as mentioned above. Similarly, in a preferred embodiment, the polypeptides of the present invention are modified to enhance their stability. It should be understood that in most cases this modification should not alter the biological activity of the interaction domain. The molecules identified in accordance with the teachings of the present invention have a therapeutic value in diseases or conditions associated with HCV infection. Alteratively, the molecules identified in accordance with the teachings of the present invention find utility in the development of more efficient anti-HCV compounds.

The level of gene expression of a reporter gene (e.g. the level of luciferase, or β-gal, produced) fused to HCV sequences within cells treated with a candidate molecule(s) can be compared to that of the reporter gene in the absence of the molecules(s). The difference between the levels of gene expression indicates whether the molecule(s) of interest influences HCV replication. The magnitude of the level of reporter gene product expressed (treated vs. untreated cells) provides a relative indication of the strength of that molecule(s) as an anti-HVC compound.

A host cell or indicator cell has been “transfected” by exogenous or heterologous DNA (e.g. a DNA construct) when such DNA has been introduced inside the cell. The transfecting DNA may or may not be integrated (covalently linked) into chromosomal DNA making up the genome of the cell. In prokaryotes, yeast, and mammalian cells for example, the transfecting DNA may be maintained on a episomal element such as a plasmid. With respect to eukaryotic cells, a stably transfected cell is one in which the transfecting DNA has become integrated into a chromosome so that It is inherited by daughter cells through chromosome replication. This stability is demonstrated by the ability of the eukaryotic cell to establish cell lines or clones comprised of a population of daughter cells containing the transfecting DNA. Transfection methods are well known in the art (Sambrook et al., 1989, supra; Ausubel et al., 1994 supra). The present invention also provides antisense nucleic acid molecules which can be used for example to decrease or abrogate the expression of the nucleic acid sequences or proteins of the present invention. An antisense nucleic acid molecule according to the present invention refers to a molecule capable of forming a stable duplex or triplex with a portion of its targeted nucleic acid sequence (DNA or RNA). The use of antisense nucleic acid molecules and the design and modification of such molecules is well known in the art as described for example in WO 96/32966, WO 96/11266, WO 94/15646, WO 93/08845 and U.S. Pat. No. 5,593,974. Antisense nucleic acid molecules according to the present invention can be derived from the nucleic acid sequences and modified in accordance to well known methods. For example, some antisense molecules can be designed to be more resistant to degradation to increase their affinity to their targeted sequence, to affect their transport to chosen cell types or cell compartments, and/or to enhance their lipid solubility by using nucleotide analogs and/or substituting chosen chemical fragments thereof, as commonly known in the art.

In general, techniques for preparing antibodies (including monoclonal antibodies and hybridomas) and for detecting antigens using antibodies are well known in the art (Campbell, 1984, In “Monoclonal Antibody Technology: Laboratory Techniques in Biochemistry and Molecular Biology”, Elsevier Science Publisher, Amsterdam, The Netherlands) and in Harlow et al., 1988 (in: Antibody—A Laboratory Manual, CSH Laboratories). The present invention also provides polyclonal, monoclonal antibodies, or humanized versions thereof, chimeric antibodies and the like which inhibit or neutralize their respective interaction domains and/or are specific thereto.

From the specification and appended claims, the term therapeutic agent should be taken in a broad sense so as to also include a combination of at least two such therapeutic agents.

The present invention relates to a kit for diagnosing or prognosing HCV infection or response to HCV to a chosen therapeutic regimen comprising a use of culturing system of the present invention. For example, a compartmentalized kit in accordance with the present invention includes any kit in which reagents are contained in separate containers. Such containers include small glass containers, plastic containers or strips of plastic or paper. Such containers allow the efficient transfer of reagents from one compartment to another compartment such that the samples and reagents are not cross-contaminated and the agents or solutions of each container can be added in a quantitative fashion from one compartment to another. Such containers will include a container which will accept the test sample (e.g. HCV nucleic acid), a container which contains the primers used in the assay to genotype chosen regions of the HCV genome, containers which contain enzymes, containers which contain wash reagents, and containers which contain the reagents used to detect the extension products.

Yet in another embodiment, the present invention relates to an assay to screen for drugs for the treatment and/or prevention of HCV infection. In a particular embodiment, such assays can be designed using cells from patients infected with HCV having a known genotype.

In accordance with the present invention, there is also provided a method for identifying, from a library of compounds, a compound with therapeutic effect on HCV infection comprising providing a screening assay comprising a measurable biological activity of a HCV protein or gene (e.g. min vitro) or measuring infectivity, (viral release etc . . . ), contacting the screening assay whether in vitro or “cellular” with a test compound; and detecting if the test compound modulates the biological activity of the protein or gene or the infectivity of the virus; wherein a test compound which modulates the biological activity or the infectivity is a compound with this therapeutic effect.

As used herein, “biological activity” refers to any detectable biological activity of a HCV gene or protein. This includes any physiological function attributable to a HCV gene or protein.

In one embodiment, the invention provides assays for screening candidate or test compounds which Interact with HCV genes or proteins.

In one embodiment, an assay is a cell-based assay in which a cell activity producing HCV is contacted with a test compound and the ability of the test compound to modulate the infectivity of HCV at different steps in the HCV complete life cycle, (e.g., attachment, entry into cells, replication, maturation etc).

The assays described above may be used as initial or primary screens to detect promising lead compounds for further development Often, lead compounds will be further assessed in additional, different screens. Therefore, this invention also includes secondary anti-HCV screens which may involve purified HCV factors.

Tertiary screens may involve the study of the Identified modulators in animal models for HCV infection. Accordingly, it is within the scope of this invention to further use an agent identified as described herein in an appropriate animal model. For example, an test compound identified as described herein can be used in an animal model to determine the efficacy, toxicity, or side effects of treatment with such an agent.

The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound”. library method; and synthetic library methods using affinity chromatography selection. The biological library approach is limited to peptide libraries, while the other four approaches are applicable to peptide, non-peptide oligomer or small molecule libraries of compounds (Lam, Anticancer Drug Des. 12: 145, 1997). Examples of methods for the synthesis of molecular libraries can be routinely found in the art for references in such methods and libraries see WO 01/38564, for example.

### BRIEF DESCRIPTION OF THE DRAWINGS

Having thus generally described the invention, reference will now be made to the accompanying drawings, showing by way of illustration a preferred embodiment thereof, and in which:

FIG. 1 shows the hepatitis C virus (HCV) genome organization;

FIG. 2 shows the hypothetical model of the HCV replication cycle;

FIG. 3 shows an experimental protocol. All experiments were performed with 1,000,000 cells/ml. T1=anti-CD3 (1 :g/:l final), IL-2 (final=200 U). T2=PHA (3 :g/:l), IL-2. T3=PHA, IL-2, SAC (1/10⁴). T4=PHA, IL-2, SAC, IL-4 (final=200 U);

FIG. 4 shows PBMC and PBLC purification from blood samples;

FIG. 5 shows the detection of HCV NS3 and NS5 proteins in cell extracts from treated PBMC from a HCV (+) patient;

FIG. 6 shows a validation that the antibody used is decorating the NS3 translated (if positive) in the replicon system and that in accordance with one embodiment of the present invention activated (A) or non-activated (NA);

FIG. 7 shows the time course of HCV-NS3 detection: PBMCs from patient MLL-001;

FIG. 8 shows the time course of HCV-NS3 detection: PBMCs from patient MLL-002;

FIG. 9 shows the detection of HCV-NS3 protein in treated (N3). PBMCs from HCV9+ donors;

FIG. 10 shows the detection of virus like particles by scanning electron microscopy;

FIG. 11 shows the electron microscopy of activated PBLs and detection of virus like particles;

FIG. 12 shows a virus partial purification;

FIG. 13 shows the detection of HCV core protein in supematant of treated PBMC from an HCV(+) patient;

FIG. 14 shows RNA quantification I (virus copies/ng total RNA);

FIG. 15 shows an infection assay co-culture system;

FIG. 16 shows infection of MT-4 cells RNA quantification 11 (virus copies/ng total RNA);

FIG. 17 shows co-culture of Huh-7 and HCV (−) PBMCs;

FIG. 18 shows co-culture of Huh-7 and HCV (+) PBMCs (SB006);

FIG. 19 shows PHA activation of PBMCs from patient SB004 (HCV is not in T cells);

FIG. 20 shows the detection of HCV (E2) on Daudi cells upon co-cultivation with infected PBMCs. Of note, Daudi cells are a B cell line;

FIG. 21 shows a comparison of different activation treatments (PBMCs from donor MLL-010). T1 PHA+IL-2. T2=SAC+IL-2. T3=T1 +T2; and

FIG. 22 shows viral RNA in cell supematant (real time RT-PCR). T1, T2, T3 are the same as for the preceding figure. Of note, further addition of IL-4 to T3 further increased activation.

FIG. 23 shows that HCV (+) and (−) strand RNA is produced de novo in activated PBLs. A) HCV-RNA was detected in PBLs from an HCV positive donor by a one step reverse transcription-polymerase-chain reaction (RT-PCR) followed by a nested PCR amplification using primers that targeted the highly conserved 5′ untranslated region (on-line material and methods). Total RNA, from either activated (P) or non-activated (N) cells, were prepared at the indicated times. RNA from Huh7 cells stably expressing the HCV replicon (Huh-Rep) (47) was used as positive control. RNA extracted from PBLs from an HCV negative donor and yeast tRNA were used as negative controls. B) Kinetics of HCV-RNA synthesis. PBLs from two positive donors, MLL-038 (Δ) and MLL-039 (◯), were stimulated by method P. RNA was extracted at the indicated time of culture and the level of HCV (−) strand RNA was determined using the Roche LightCycler system. RNA levels were normalized against GAPDH and are reported as a fold variation relative to the amount of (−) strand RNA in non-treated PBLs. C, D) Bromo-uridine incorporation into de novo synthesized RNA was detected in by immunofluorescence using an anti-bromodeoxyuridine antibody. C) HCV positive donor MLL-069. D) HCV negative donor.

FIG. 24 shows that HCV proteins are produced in activated PBLs. PBLs were stimulated using method P. Protein extracts were prepared following five days of activation. A) Extracts from either treated (P) or non-treated (N) PBLs, from donor SB-1 were run side by side with extracts from Huh-7 cells expressing the HCV replicon (Huh-Rep) (47). “NS3 was detected using a polyclonal antibody). Extracts from PBLs, either treated (P) or non-treated (N), from a HCV negative donor were run side by side with extracts from donor SB6. NS3 was detected using monoclonal antibody 1G3D2. C) Extracts from Huh-7 cells and Huh-Rep, were run side by side with extracts, either treated (P) or non-treated (N), from an HCV negative and positive donor. NS5B was detected using a monoclonal antibody such as 5B-10 (IFA). D) Extracts from either treated (P or A) or non-treated (N) PBLs from different HCV positive donors were run side by side with extracts from an HCV negative donor, Huh-7 or Huh-Rep cells. NS3 was detected using monoclonal antibody 1G3D2. E) Kinetics of NS3 synthesis following PBLC stimulation by methods P, S and PS. Extracts were prepared on the indicated days and NS3 was detected using monoclonal antibody 1G3D2. F, G, H) Kinetics of NS3 accumulation in donors MLL-001, MLL-002 and MLL-010 after stimulation using method P. Extracts were prepared on the indicated days. An extracts from non-treated cells was prepared either on day 3 (F and G) or on day 2 (H). NS3 was detected using anti-NS3 monoclonal antibody IG3D2 (F and G) or with an NS3 rabbit antiserum (H). Actin or a non-specific band, LC, identified by antibody 1G3D2, were used as loading controls. I, J, K.) siRNA silencing of HCV RNA. Core-siRNA or a non-specific RNA sequence (nsRNA) were electroporated into PBLs three days after stimulation. Proteins and RNA were extracted 48 hr later. I) NS3 and NS5B were detected with NS3 rabbit antiserum and 5B-3B1 monoclonal antibody (48), respectively. Actin was used as an internal control. J) RNA levels were quantified by real-time PCR (method I, materials and methods). Absolute copy number of the HCV (+) strand transcripts (Δ) and the amount of GAPDH (◯) RNA are shown. K) HCV RNA amounts were normalized against GAPDH. The ratio of HCV/GAPDH was determined for the nsRNA and assigned an arbitrary value of 100. The Core-siRNA HCV/GAPDH ratios are expressed relative to the negative control.

FIG. 25 shows that HCV Core protein was detected by indirect Immunofluorescence in day 3 stimulated (P) PBLs from MLL-059, using the RR8 polyconal antibody. Stimulated PBLs from an HCV negative donor were used as a control.

FIG. 26 shows that HCV is released from activated HCV positive PBLs. A, B) Supematant from stimulated PBLs (method P) was collected and sedimented through a 20% sucrose cushion. A) Sedimented proteins were resolved by SDS 15%-PAGE, transferred to a nitrocellulose membrane (overnight, 30V) and detected using MAB255P monoclonal anti-core antibody (Maine Biotechnology Services, Inc.). HCV (−) corresponds to the negative control. B) RNA was analyzed by nested RT-PCR. RNA from Huh-Rep was used as a positive control. RNA from yeast tRNA, Huh-7, and -an HCV negative donor were used as negative controls. C) PBLs from donor. SB-5 were stimulated using methods B, P, and PS. Five days following activation, the supematant was collected and sedimented through a 20% sucrose cushion. The quantity of HCV RNA was determined by real-time RT-PCR on the ABI Prism 7700 Sequence Detection System. D) Following metabolic labeling (35S Met/Cys) of PBLs from donor MLL-035, the supernatant was sedimented through a 20% sucrose cushion. The sediment was resuspended and analyzed by a flotation gradient. Collected fractions were resolved on a SDS-15%, PAGE, transferred to a nitrocellulose membrane and exposed to a Kodak Biomax MR film. E) Fractions were concentrated and HCV E2 glycoprotein visualized by Western bolting using monoclonal anti-E2 1864 (450470AA) antibody. F) RNA was extracted from the gradient fractions of FIG. 4E. and the absolute quantity of HCV1 RNA was determined by real time RT-PCR.

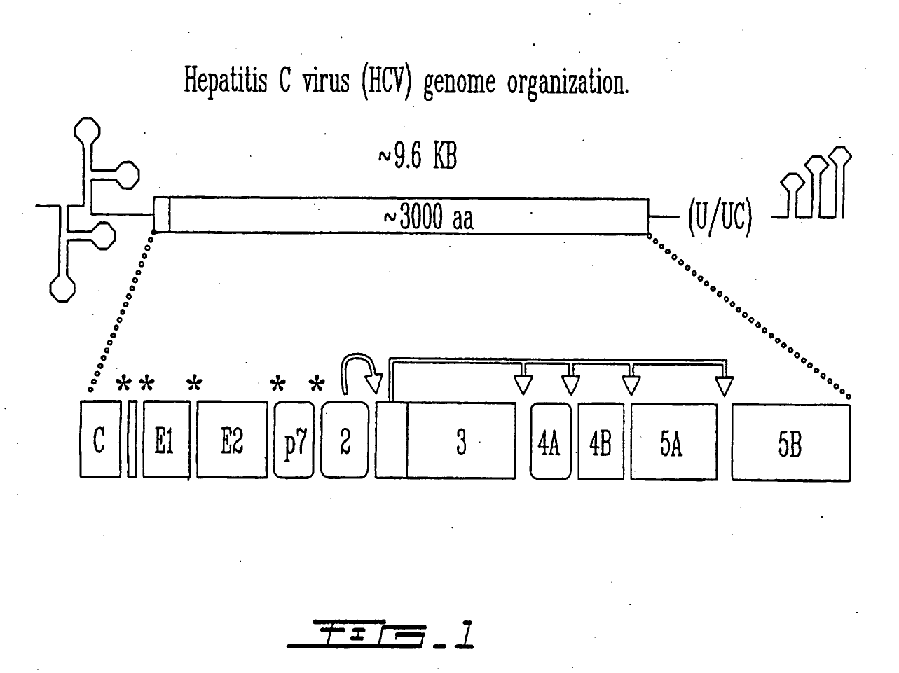

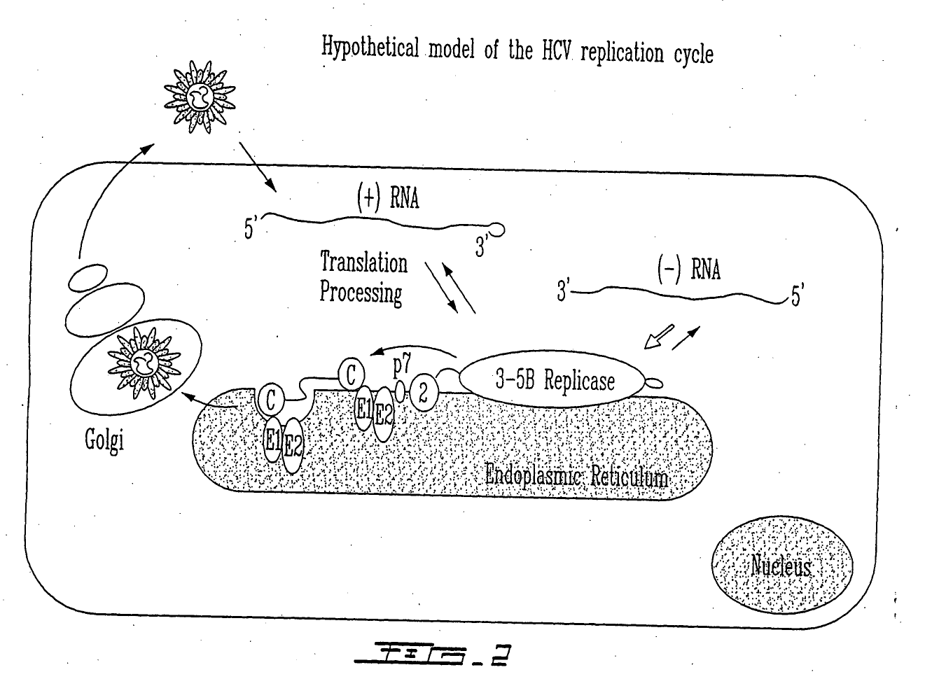

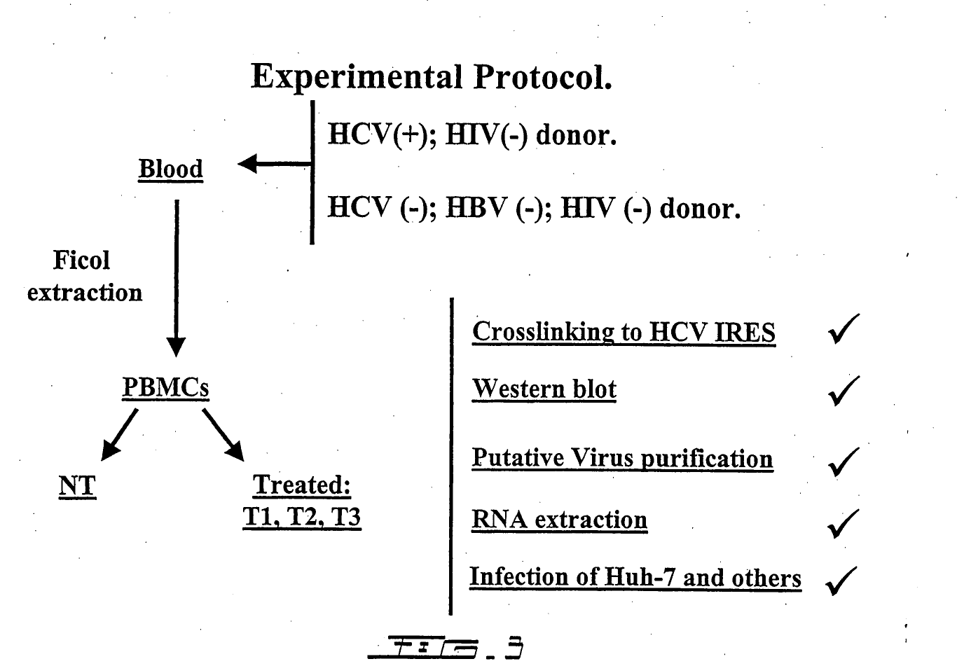

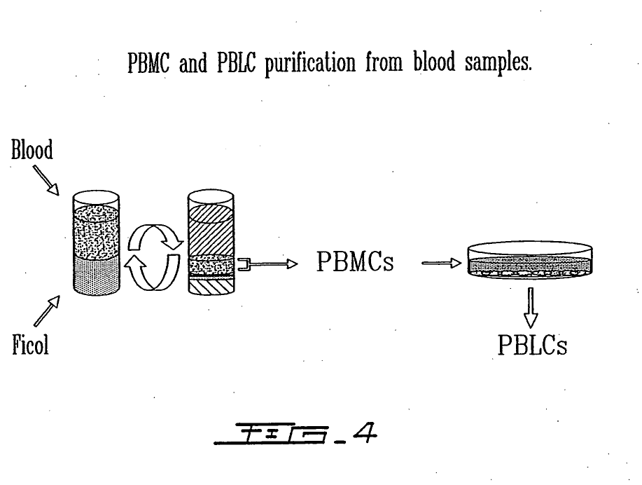

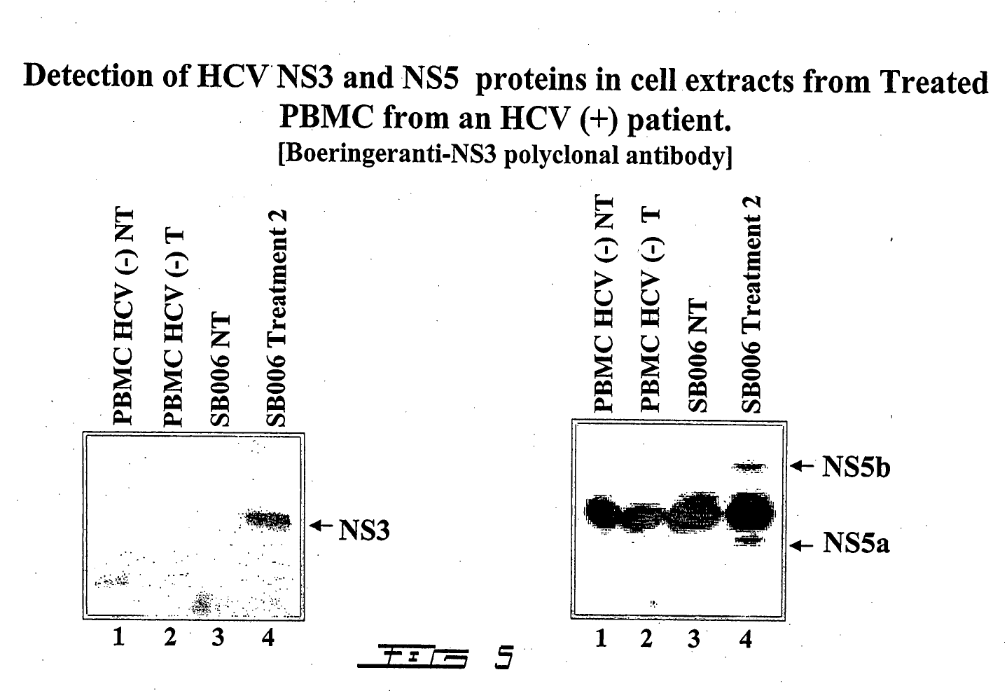

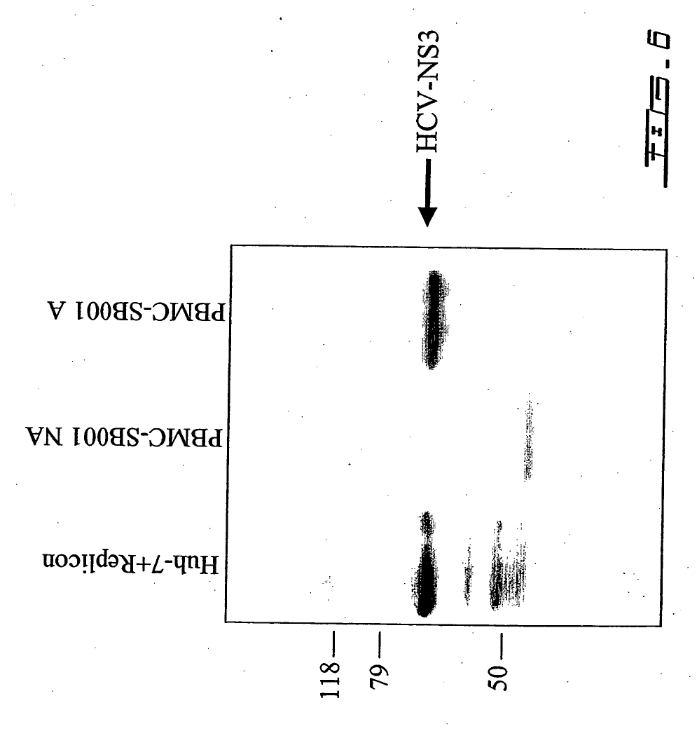

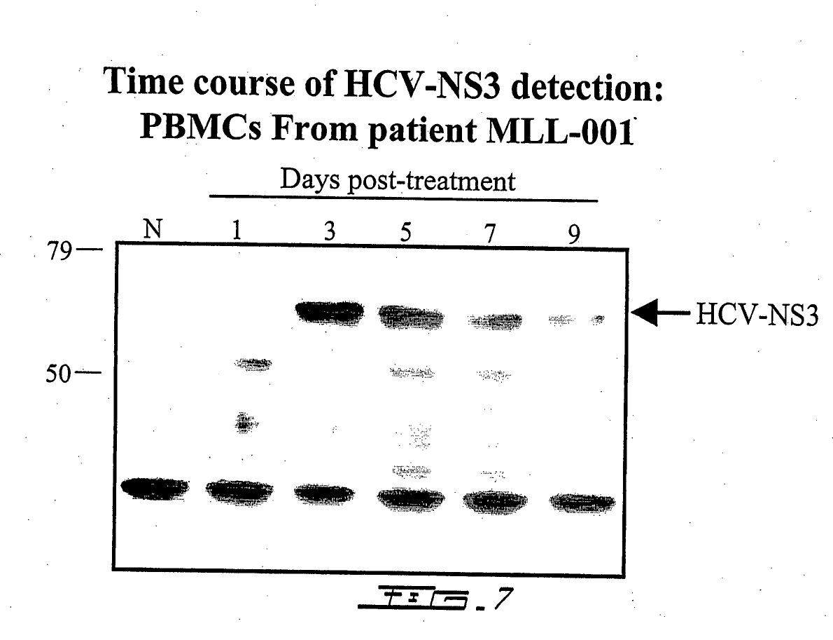

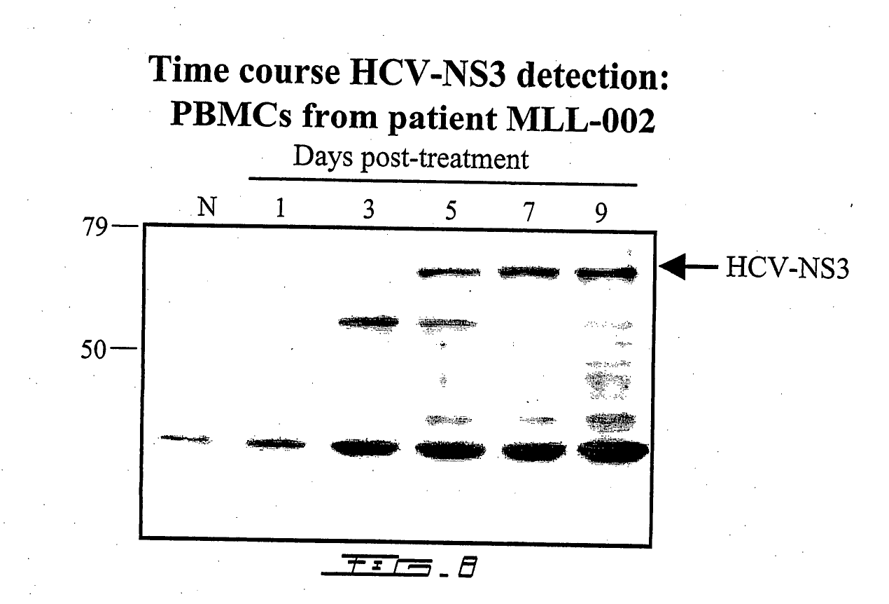

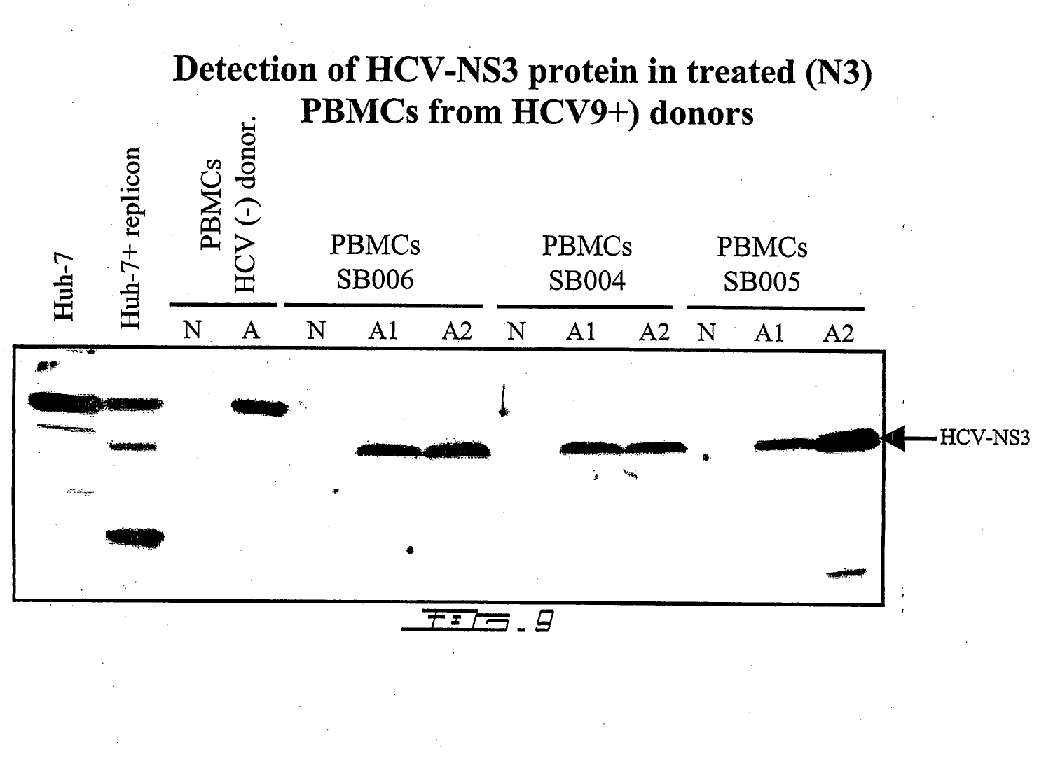

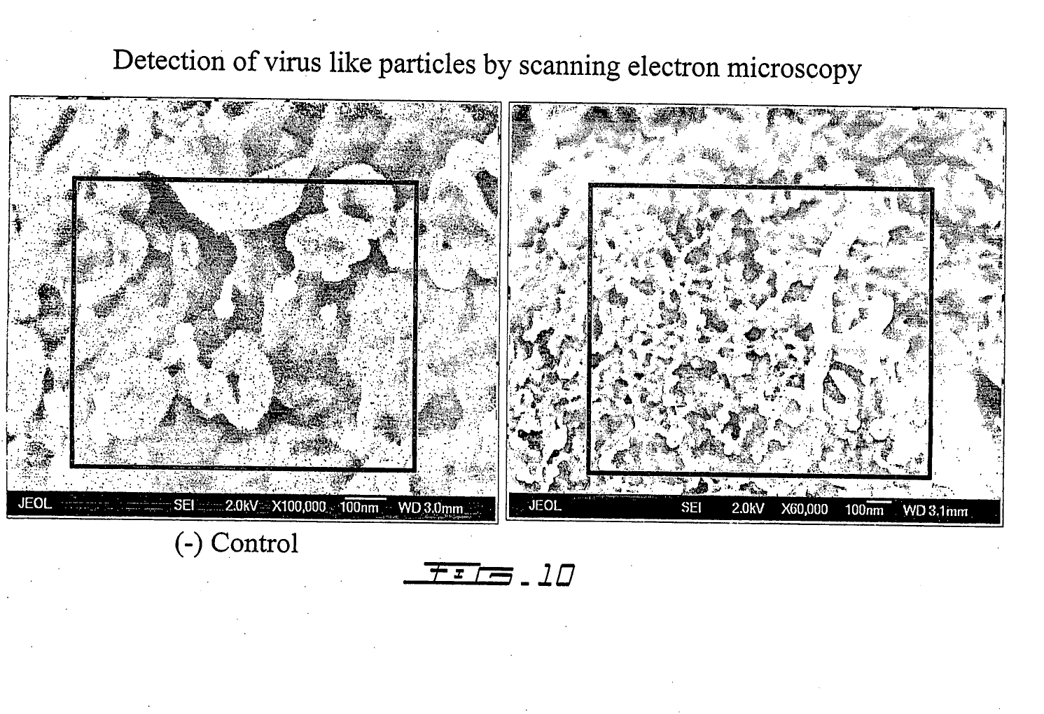

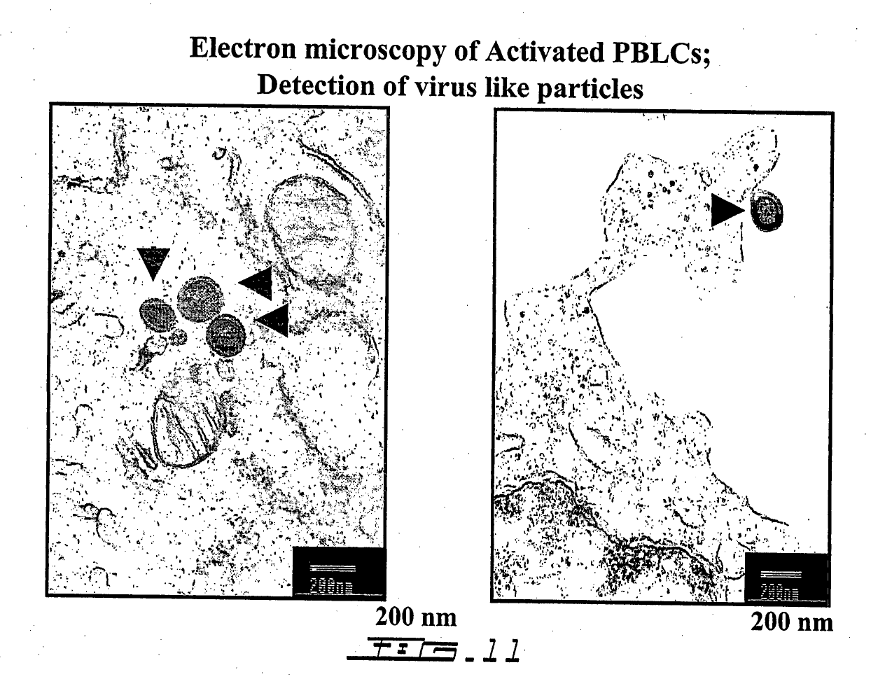

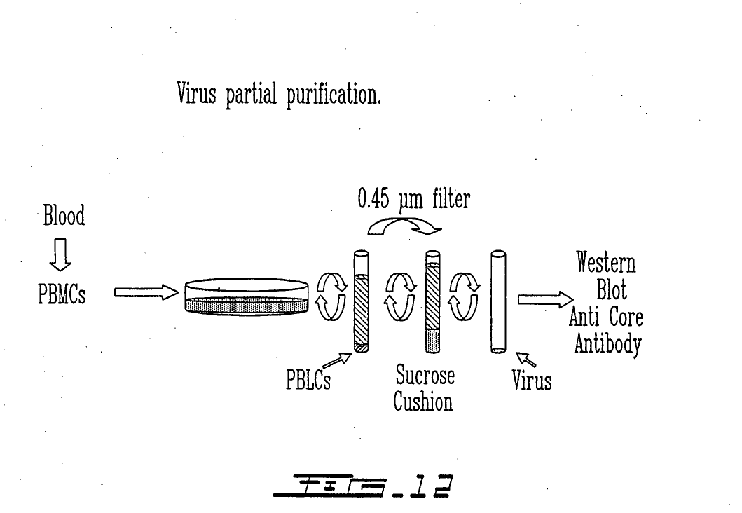

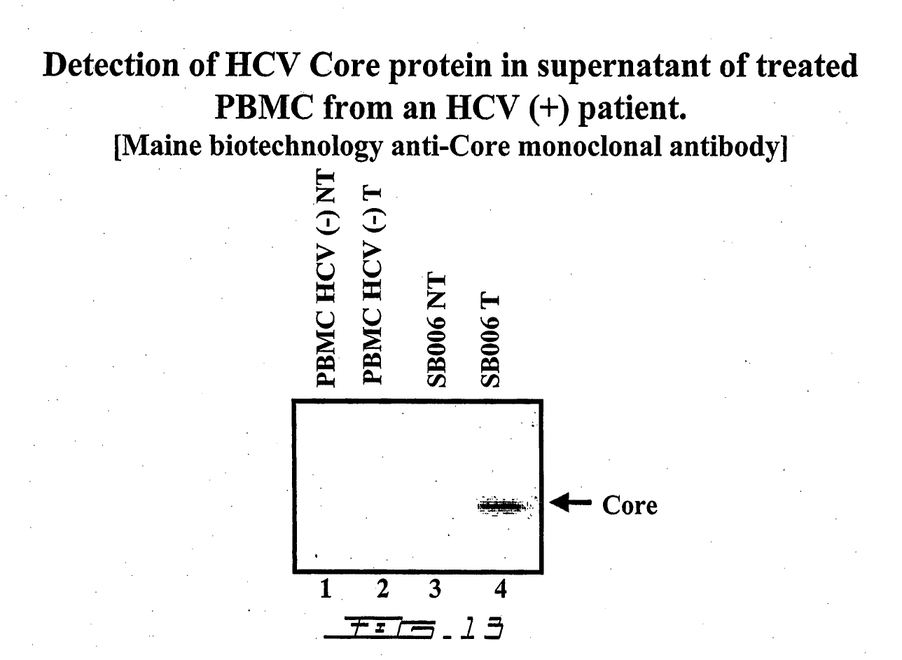

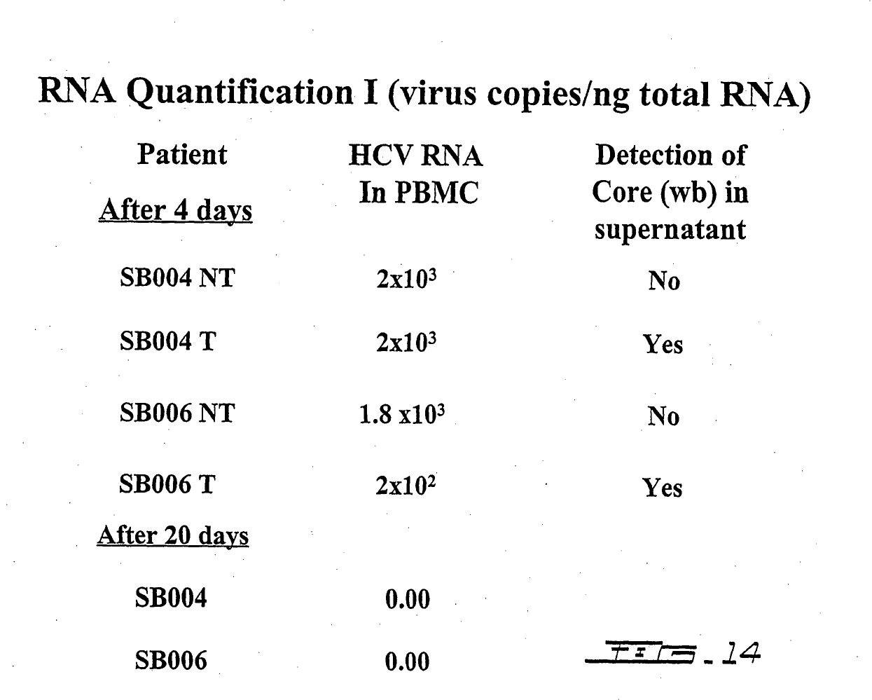

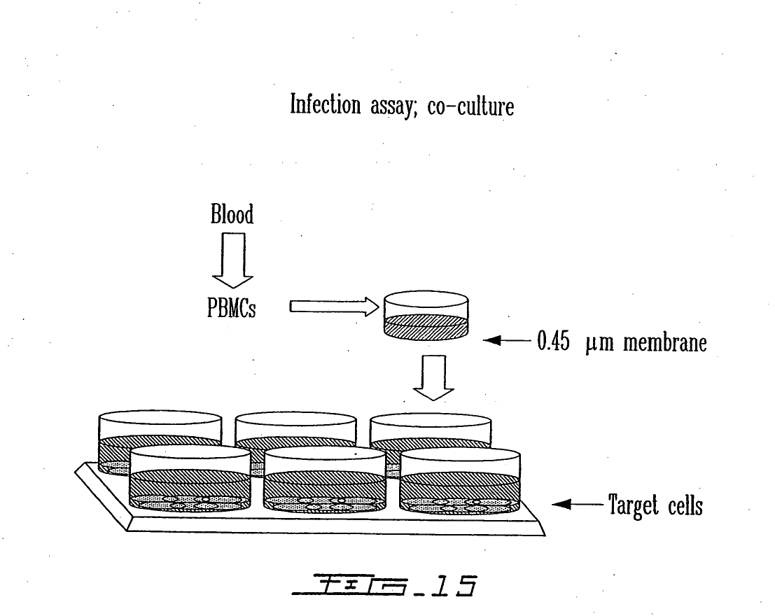

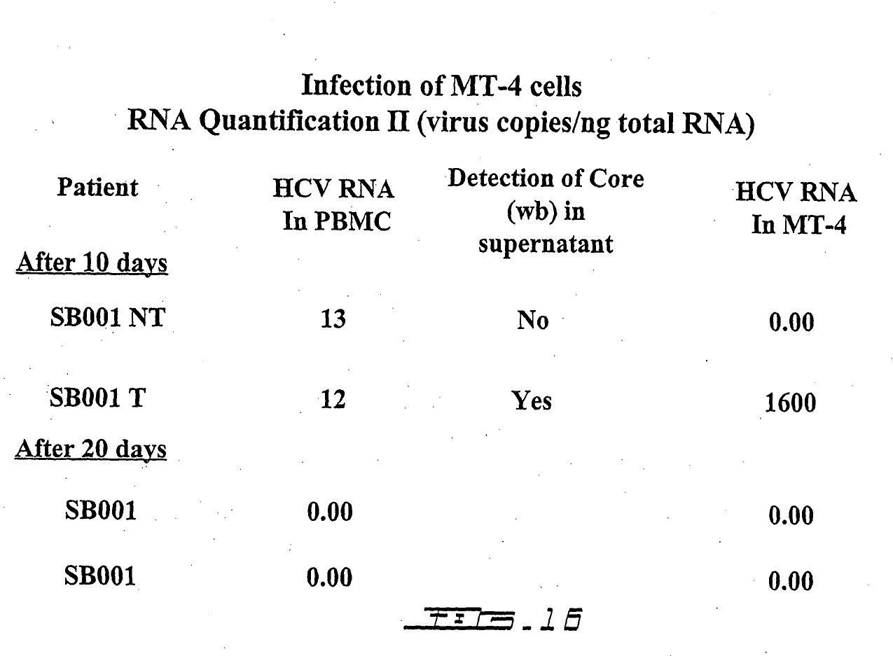

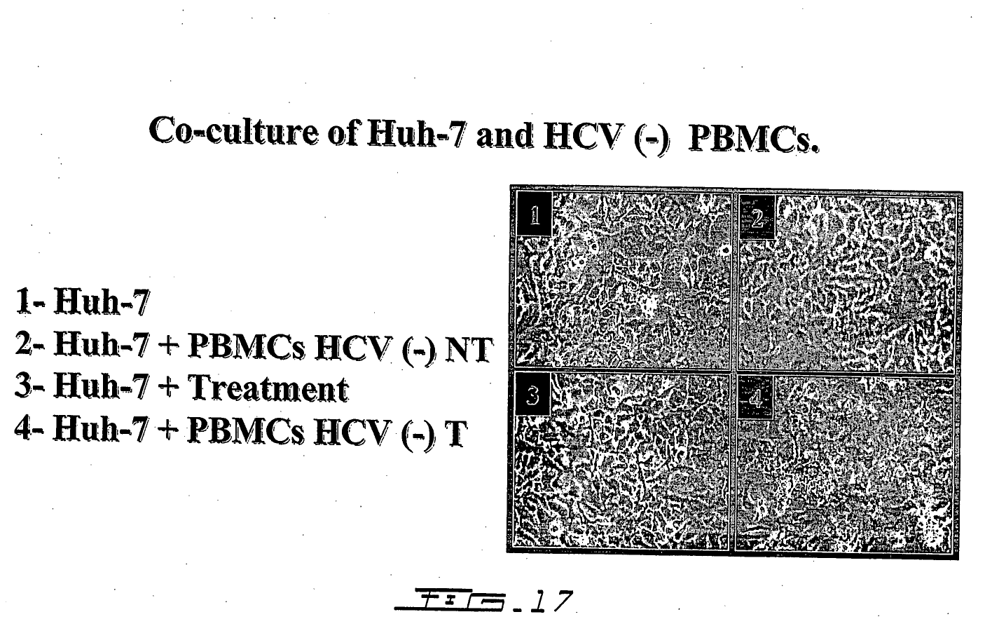

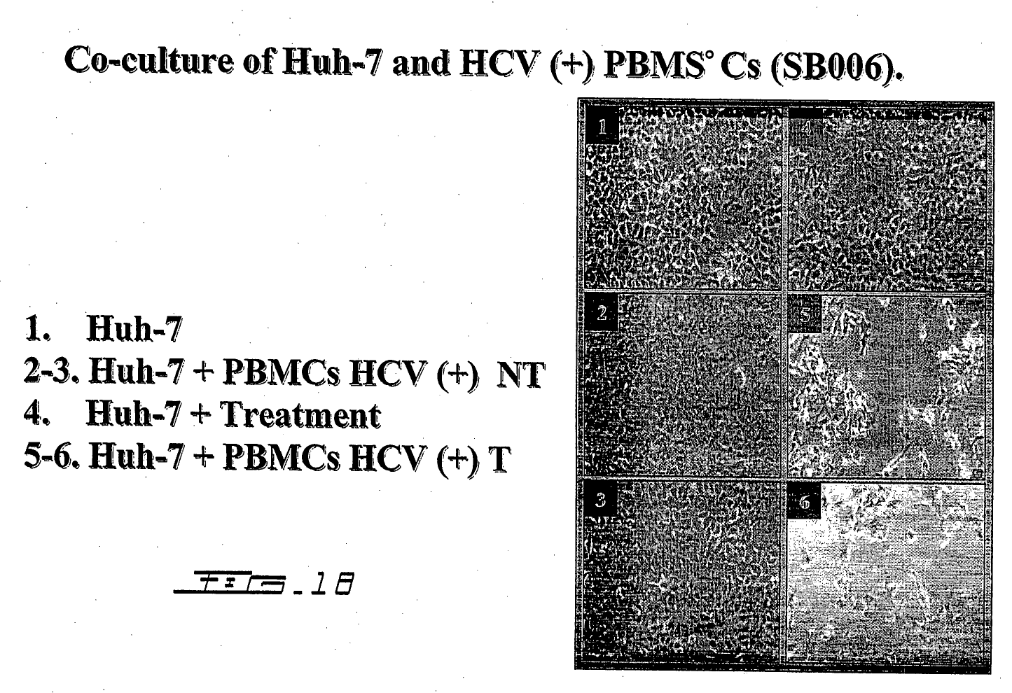

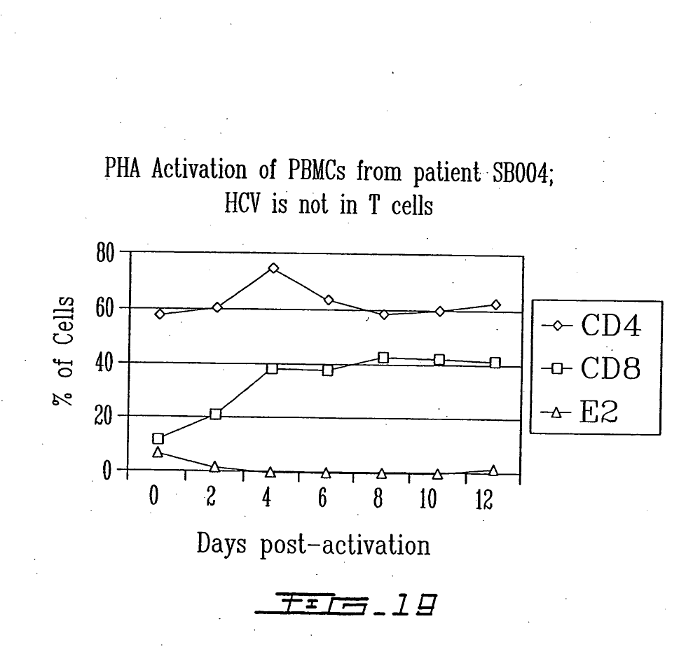

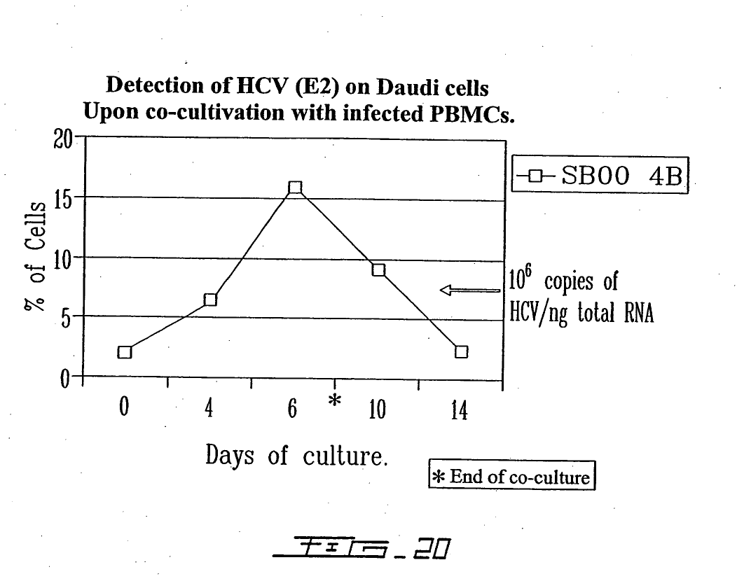

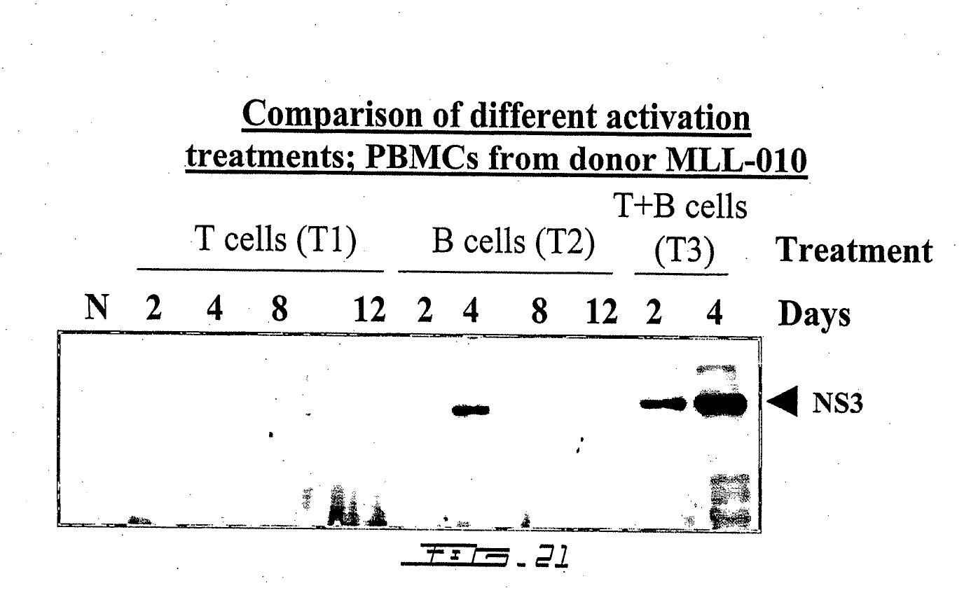

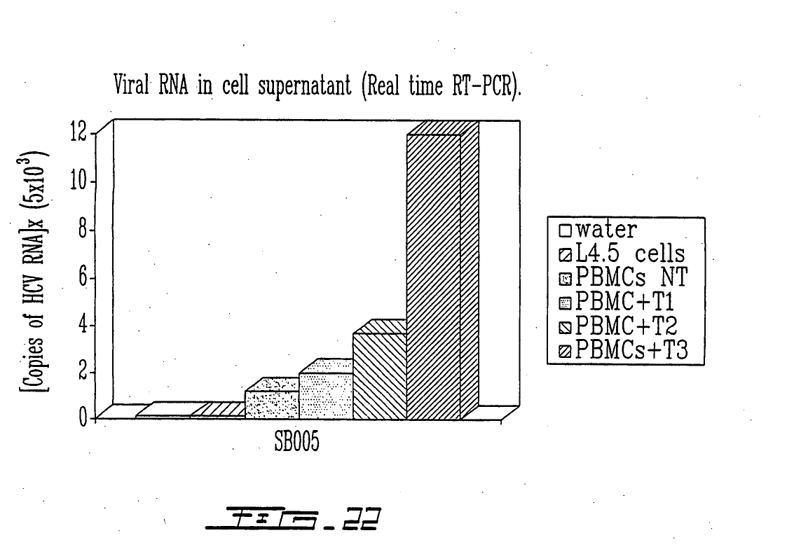

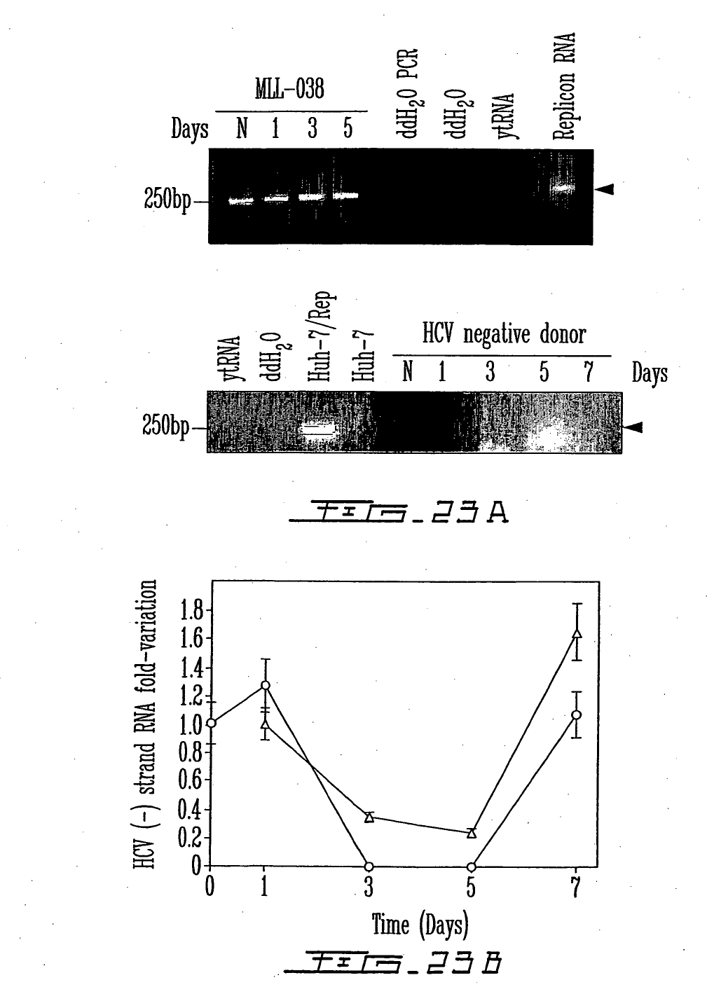

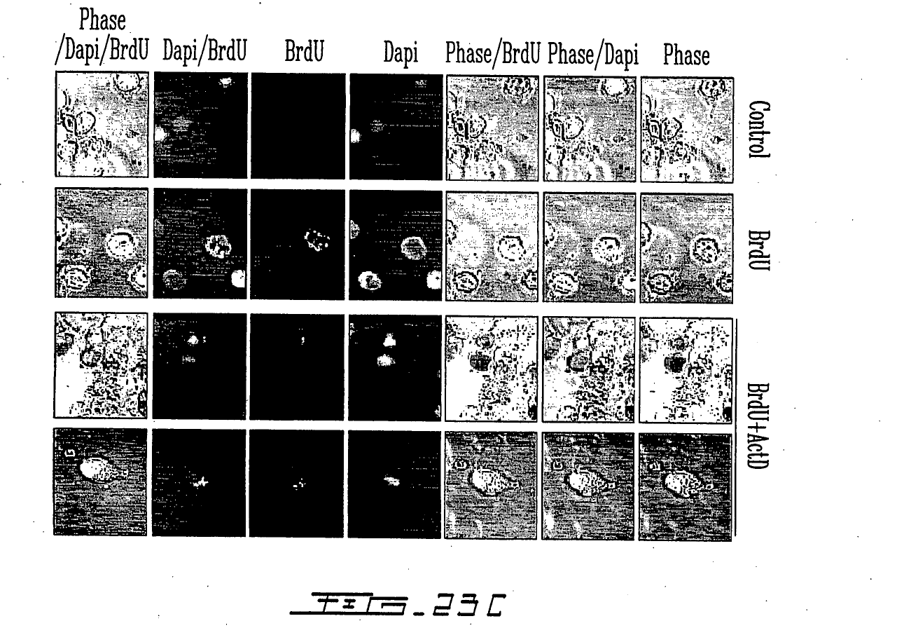

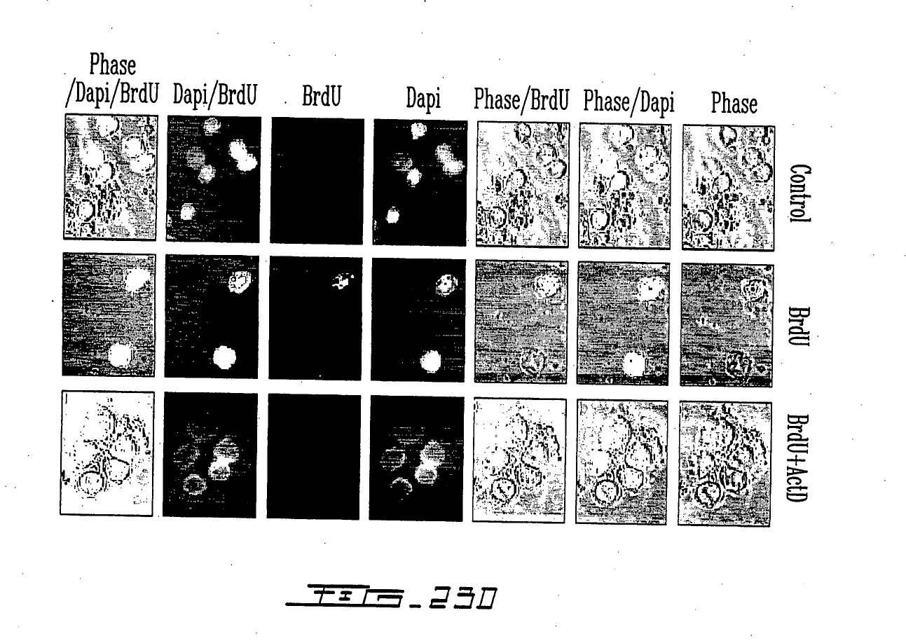

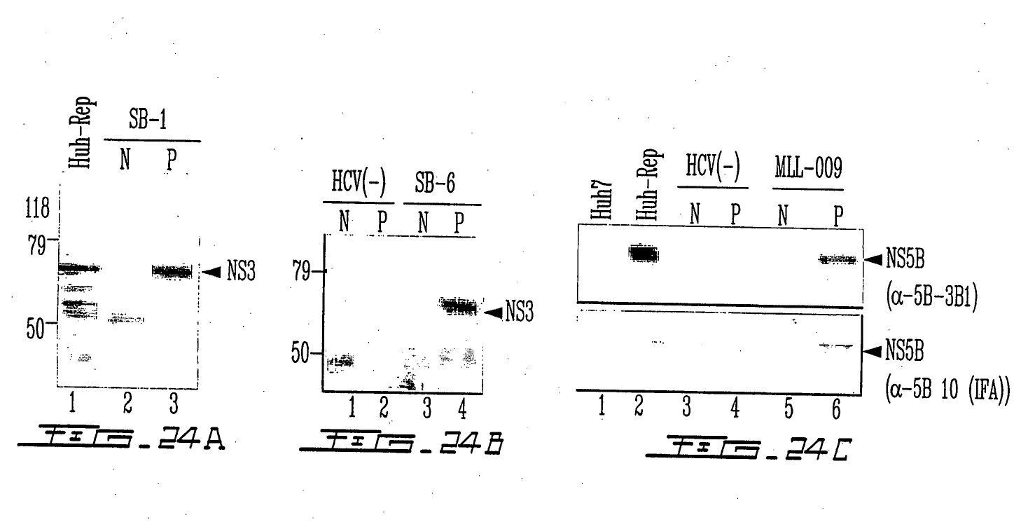

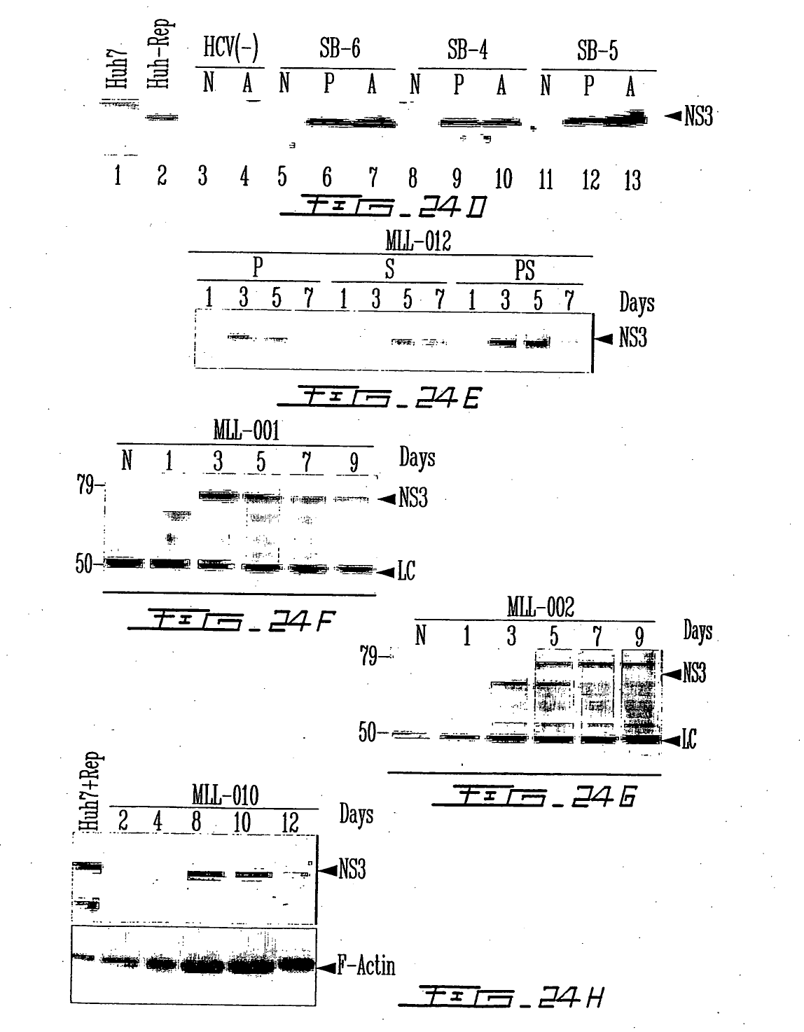

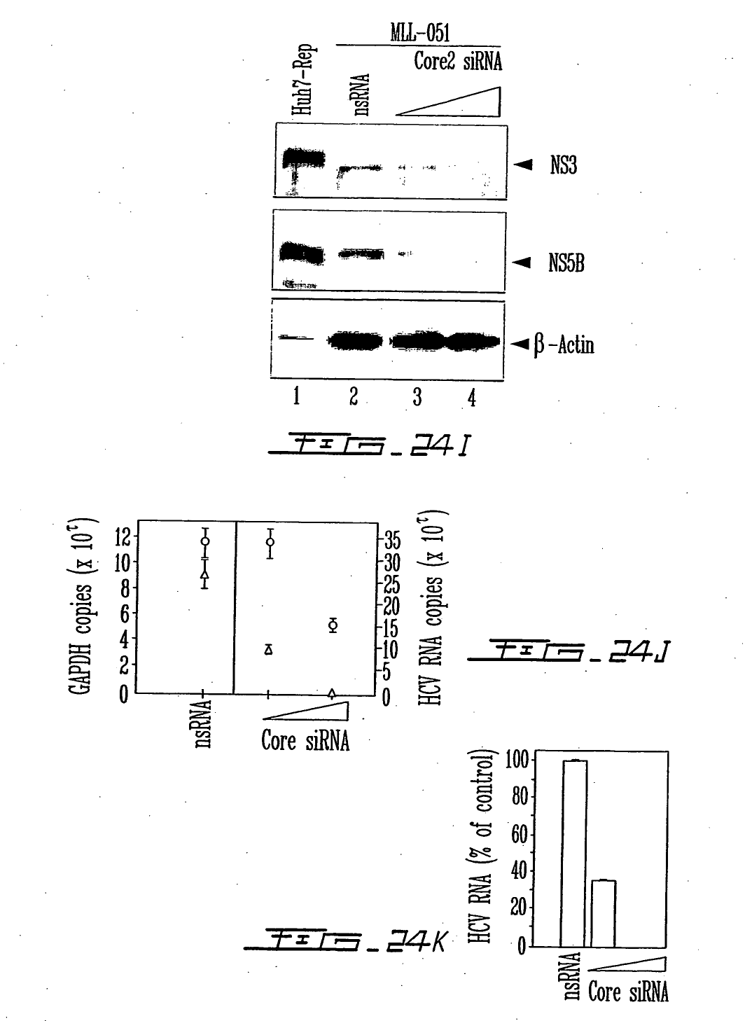

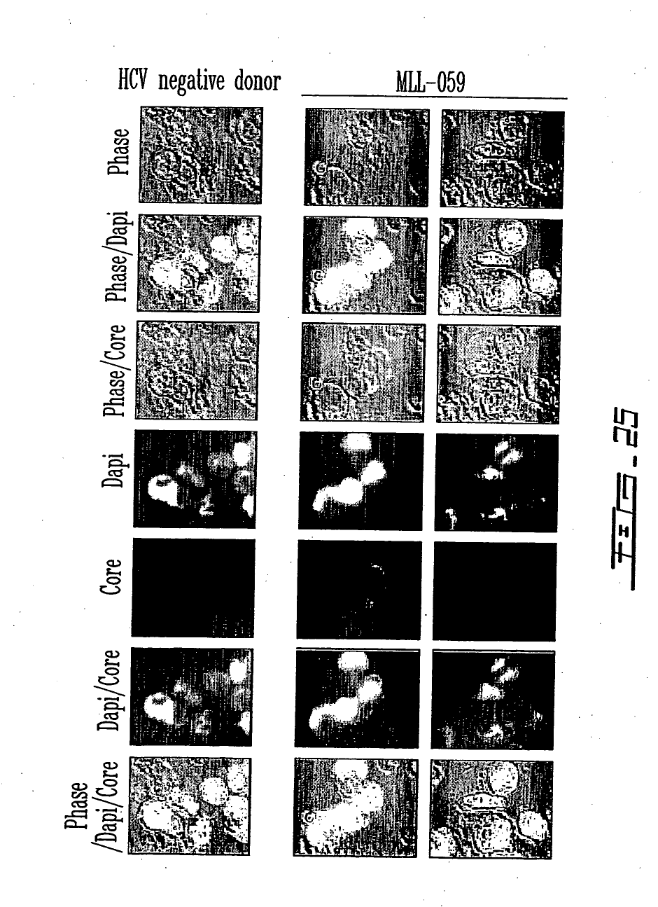

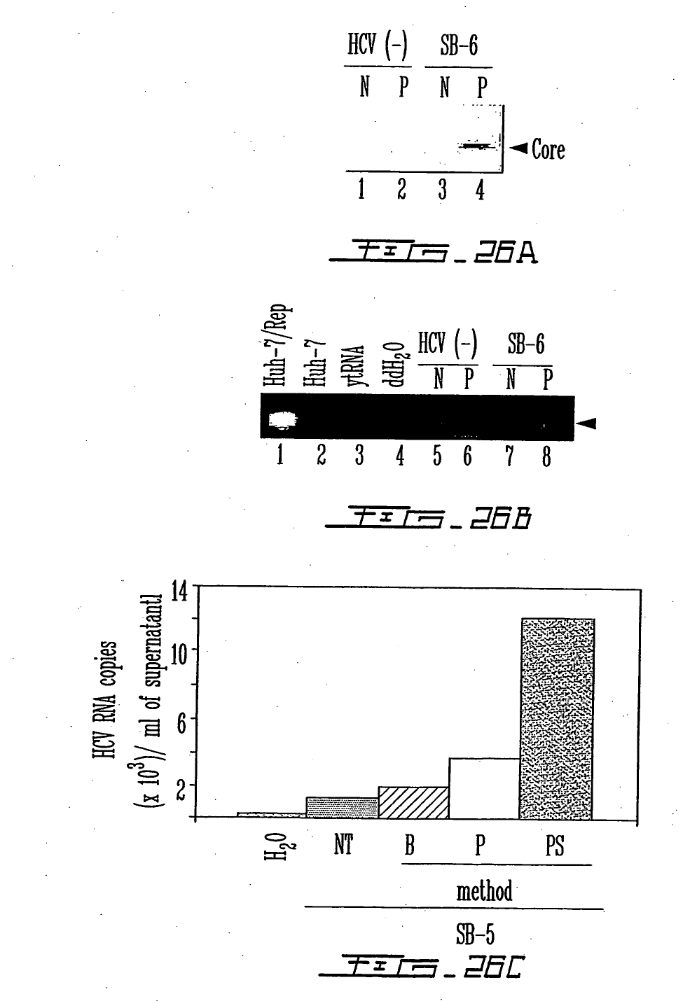

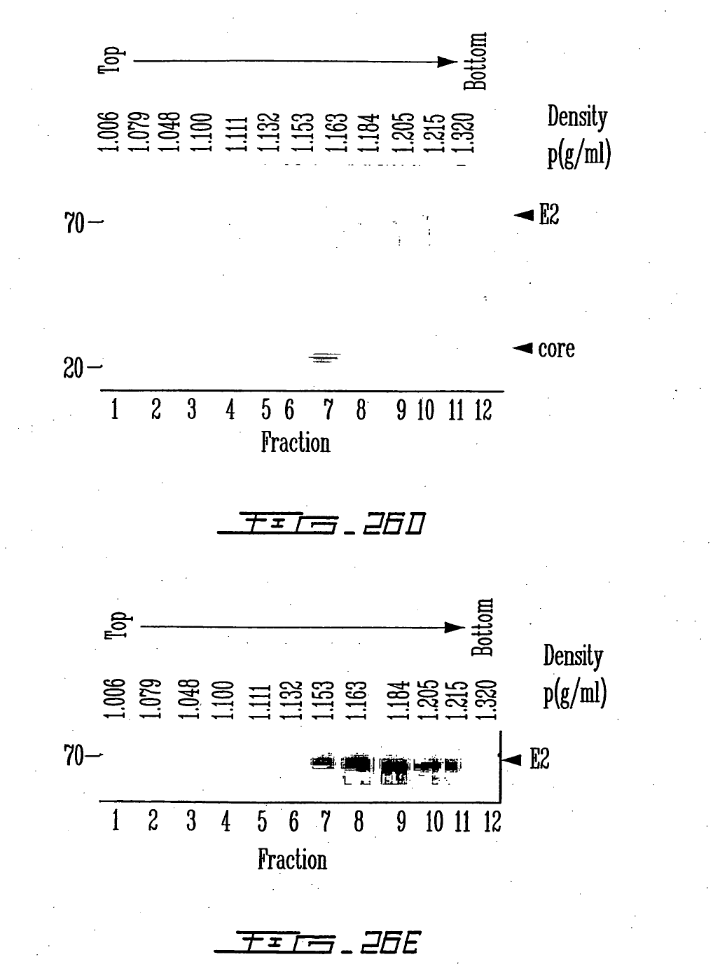

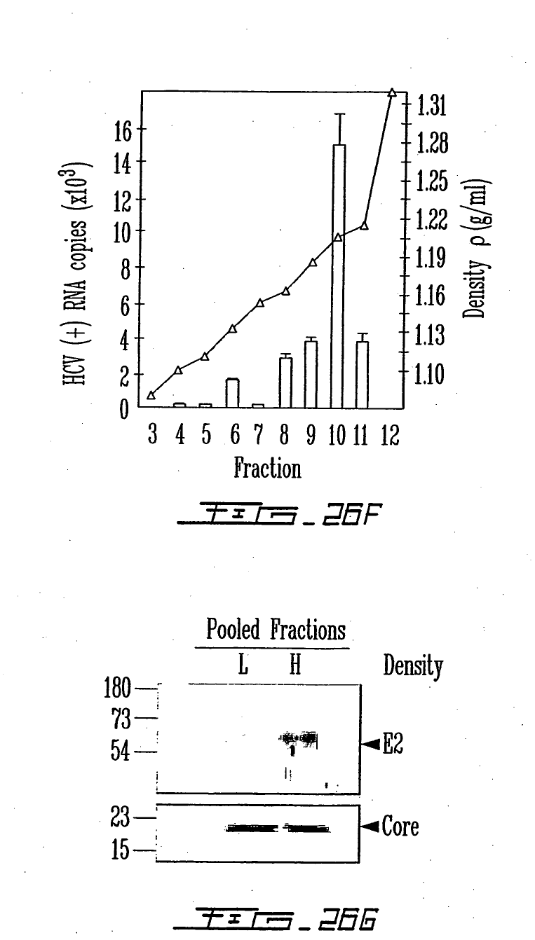

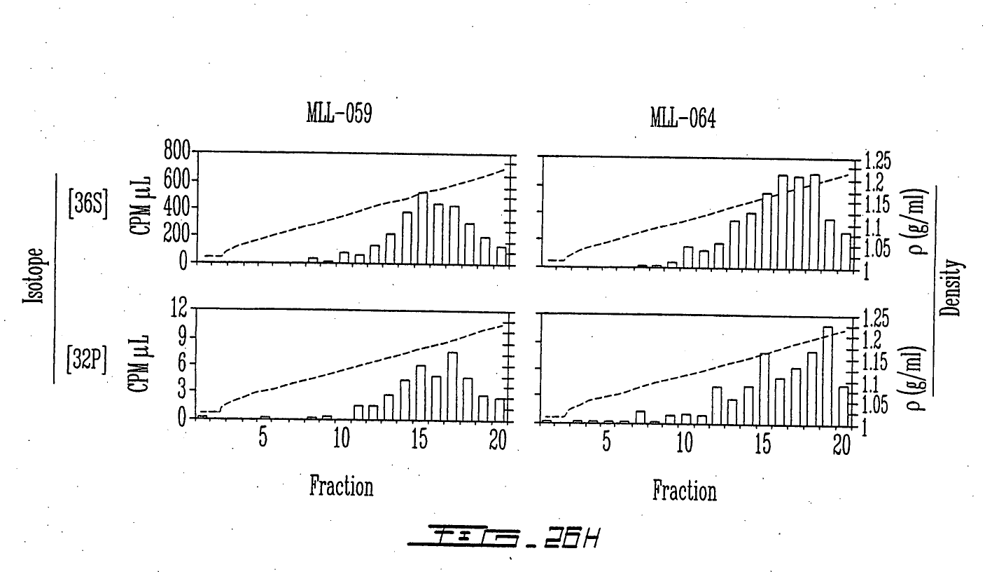

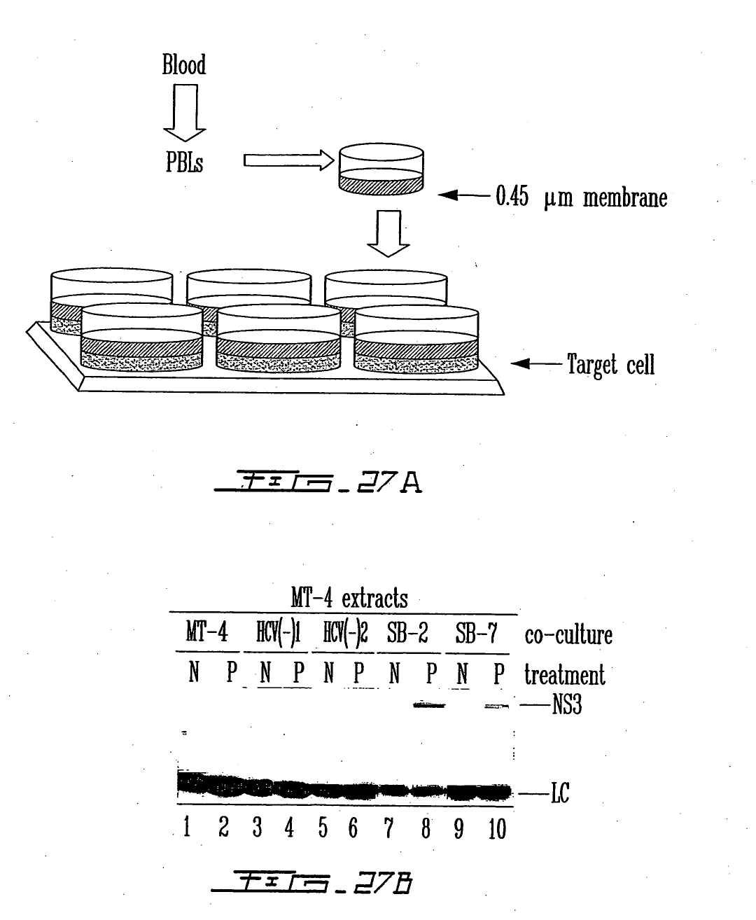

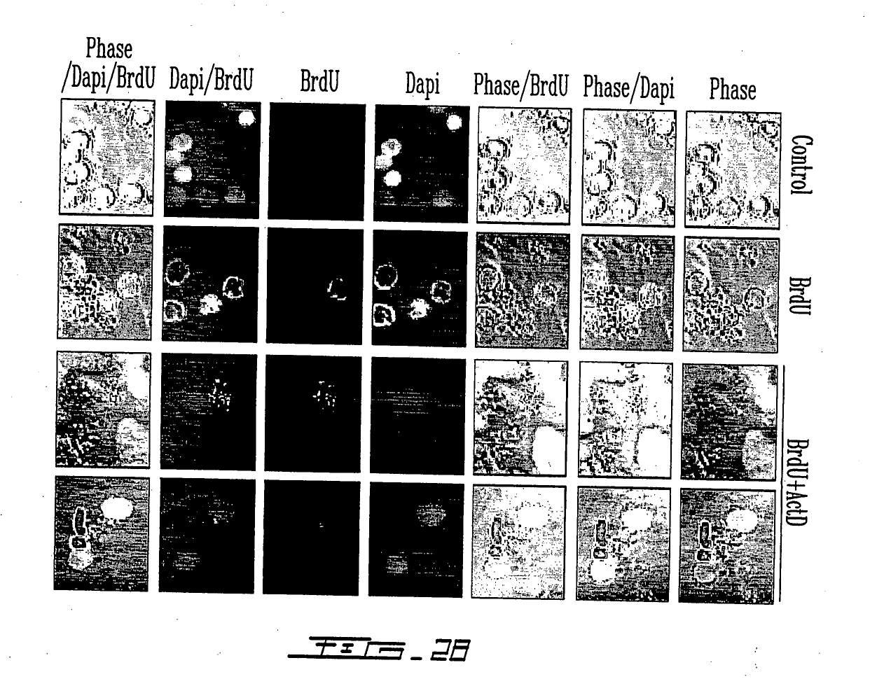

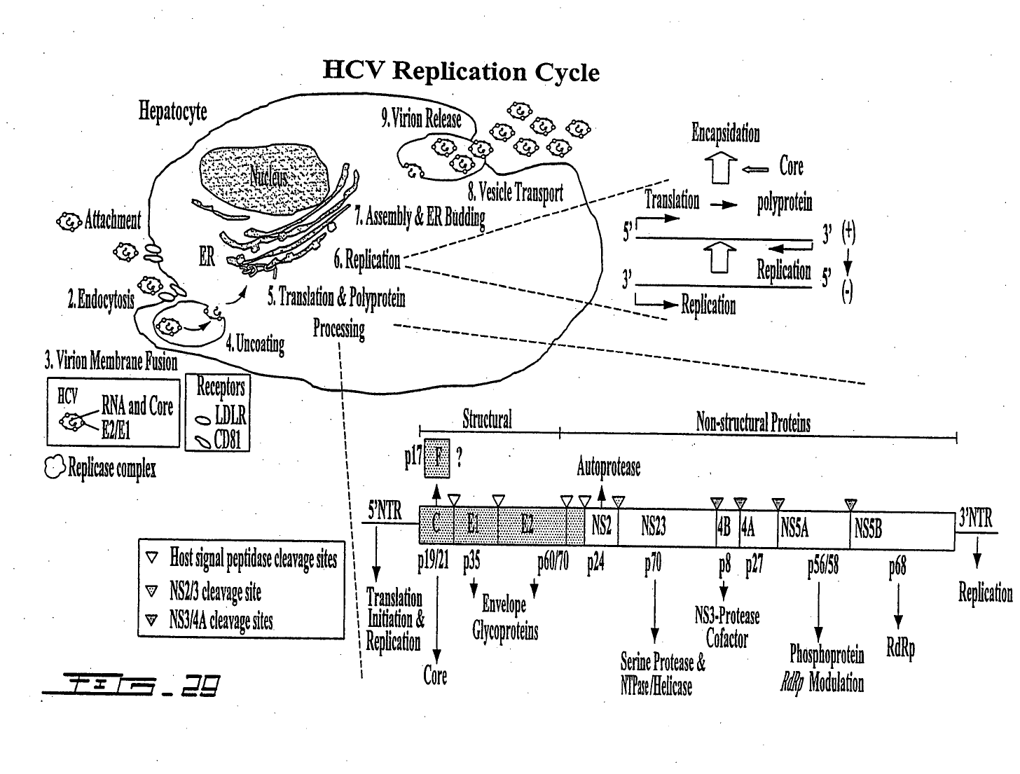

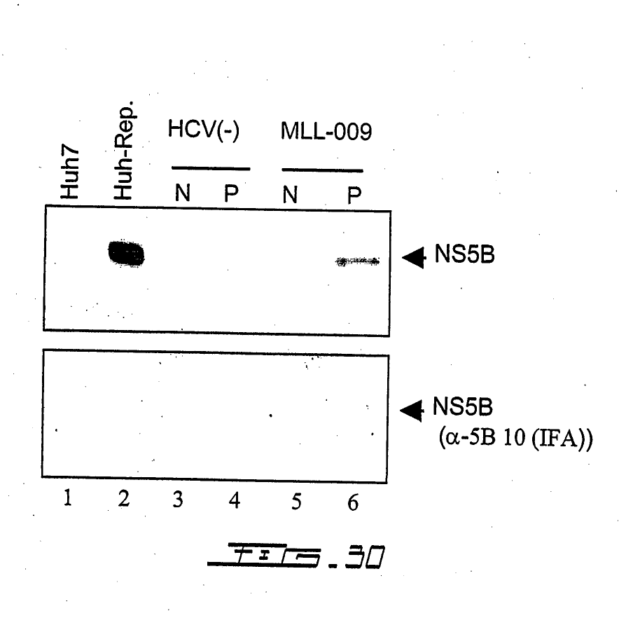

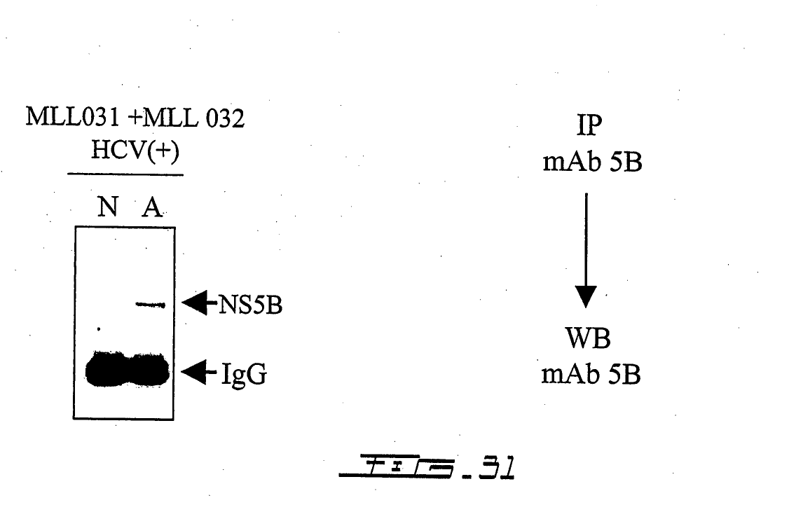

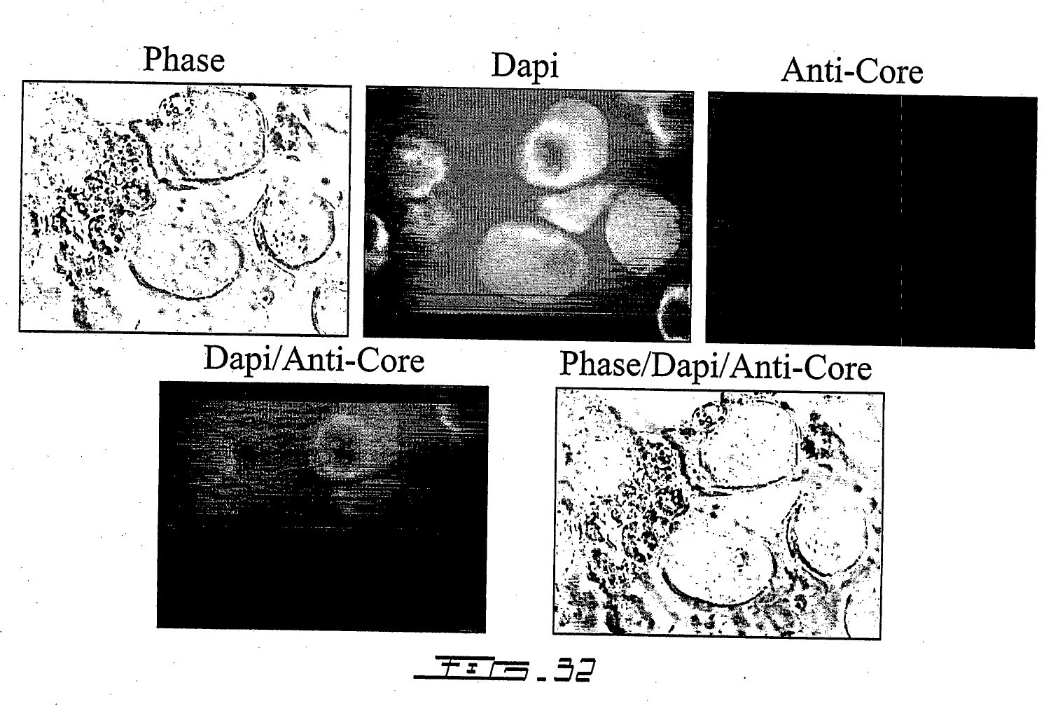

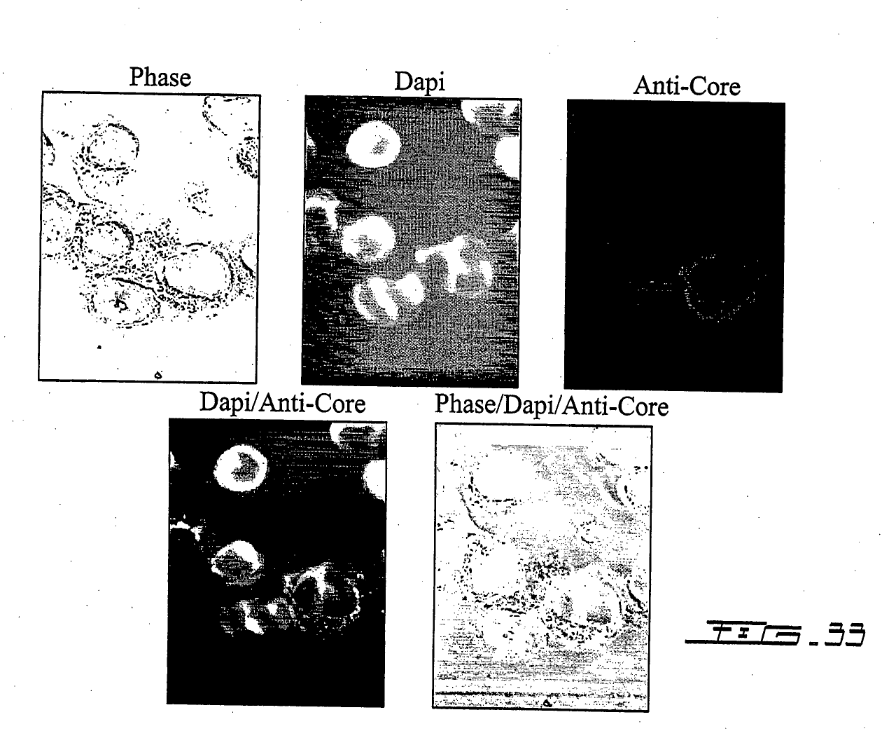

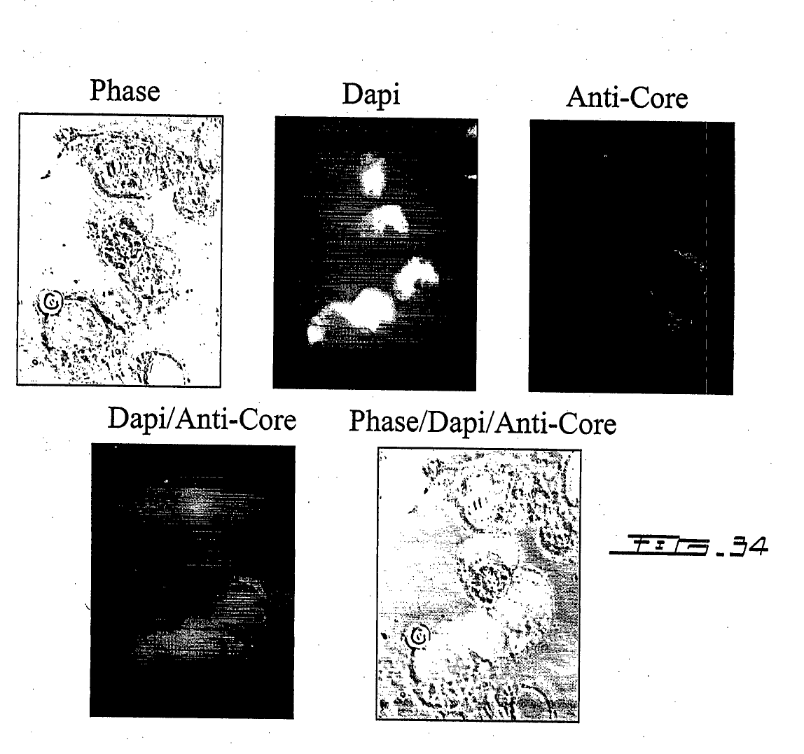

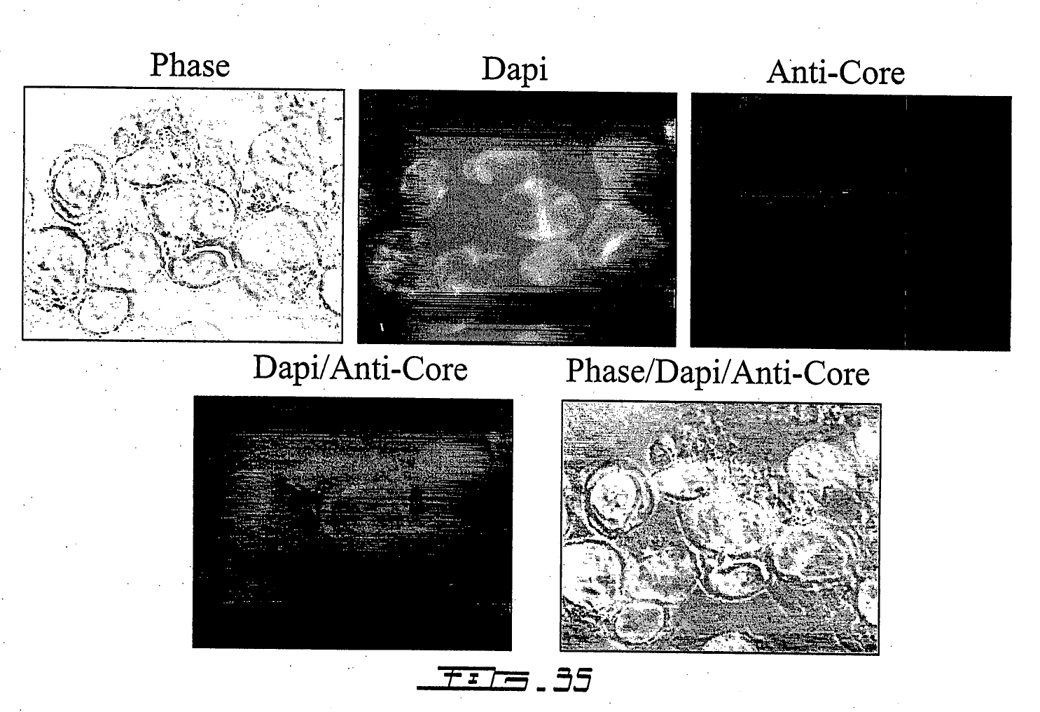

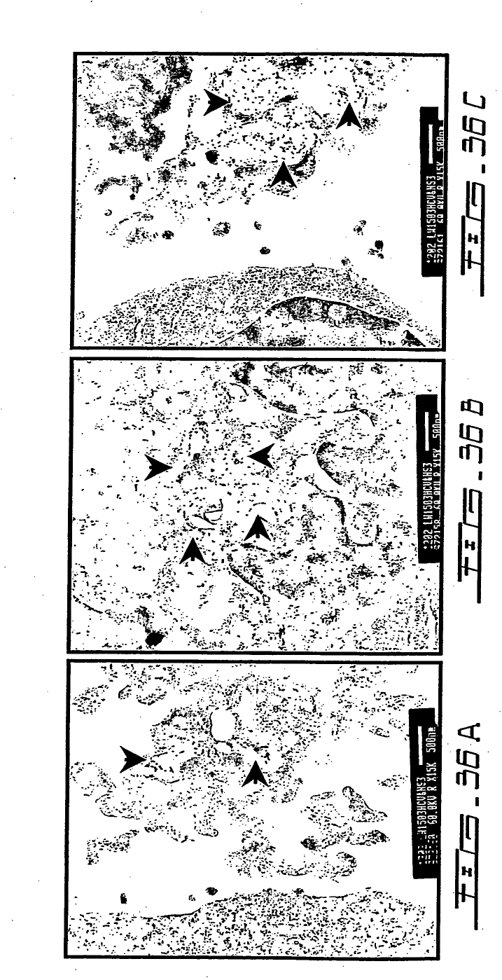

G) Fractions 1-4 (L) and 5-11 (H) from the flotation gradient were concentrated and pooled. Proteins were resolved on a SDS-15% PAGE. HCV E2 glycoprotein was detected using monoclonal antibody 1864 (450470AA). Core protein was visualized using monocloal anti-core 515S (2040M) antibody. H) Activated PBLs from donors MLL-059 and MLL-064 were metabolic labeled for 12 h with ³⁵S-Met/Cys or ³²P-orthophosphate. Supernatants were sedimented through a 20% sucrose cushion. The sediments were resuspended and analyzed by a flotation gradient. The amount of incorporated radioactivity in each fraction of the gradients was determined in a Beckman LS 6500 scintillation counter.

FIG. 27 shows that virus released from activated HCV positive PBLs is infectious. A) Schematic representation of the co-culture chambers used in these experiments. B) MTS cells were co cultured with either treated (P) or non-treated (N) MT4 cells, PBLs from two HCV negative donors or PBLs from donors SB-2 or SB-7. Extracts were prepared following six days of co-culture. NS3 was detected using monoclonal anti-NS3 antibody 1G3D2. LC indicates a non-specific band used as a loading control.

FIG. 28 shows Bromo-uridine incorporation into de novo synthesized RNA and detected by immunofluorescence using an anti-bromodeoxyuridine antibody in PBLs from donor MLL-065.

FIG. 29 shows the HCV replication cycle.

FIG. 30 shows the detection of HCV protein by immunoprecipitation.

FIG. 31 shows the detection of HCV protein by Western Blot;

FIG. 32 shows immunofluorescence of HCV (−) Control Polyclonal-anti Core RR8.

FIG. 33 shows immunofluorescence of MLL-059 Anti-Core RR8.

FIG. 34 shows immunofluorescence of “MLL-059 Anti-Core RR8.

FIG. 35 shows immunofluorescence of MLL-059 Anti-Core RR8.

FIG. 36 shows immuno-electronmicroscopy of HCV protein using an anti NS3 antibody.

FIG. 37 shows electron microscopy of cells showing HCV viral particle assembly.

FIG. 38 shows an embodiment of a scheme for virus partial purification.

FIG. 39 shows density determination of HCV viral particles purified according to FIG. 38.

FIG. 40 shows that PBMC generate two HCV subpopulations that can be partially purified by density gradient.

FIG. 41 shows an embodiment of a protocol to assess infectivity of isolated HCV.

FIG. 42: alpha IFN. PBLs from donor MLL-0015 were stimulated using PHA and Sac in presence or absence of 1000 IU/ml of alfa-Interferon. HCV NS3 protein was used as a readout of viral replication. NS3 was detected using monoclonal antibody 1G3D2.

FIG. 43: PBLs from an HCV (+) donor were stimulated using PHA in presence or absence of 100 μM of candidate compound X.

Other objects, advantages and features of the present invention will become more apparent upon reading of the following non-restrictive description of preferred embodiments with reference to the accompanying drawings which is exemplary and should not be interpreted as limiting the scope of the present invention.

### DESCRIPTION OF THE PREFERRED EMBODIMENTS

The existence of extrahepatic reservoirs of hepatitis C virus (HCV) replication remains controversial. Several groups have described the presence of hepatitis C virus (HCV) genomic sequences (plus-strand) and replicative intermediate (minus-strand) in peripheral blood mononuclear cells (PBMC). The association of HCV RNA with peripheral blood leukocytes has been documented since 1992 (Proc. Natl. Acad. Sci. USA, 1992, 89:5477; J. Virol. 1993, 67:1953; Hepatology 1996,-23:205, J. Virol., 1996, 70:3325-9; J. Virol. 1996, 70:7219-23; Antiviral Research 2001, 52:1-17). However, the specificity of the methods used in these studies has been questioned. More recent reports, which used an optimized negative strand-specific reverse-transcriptase polymerase chain reaction (RT-PCR) assay, detected negative-strand HCV only in PBMC taken from post-transplant or human immunodeficiency virus (HIV)infected HCV patients, and not in PBMC from typical patients with chronic HCV infection. Of note, a number of studies have also reported that human B and T cell lines are capable of supporting a productive infection. However, the data supporting viral production was only based on RNA detection (Proc. Natl. Acad. Sci. USA, 1992, 89:5477; J. Virol. 1993, 67:1953; Hepatology, 1996, 23:205; J. Virol., 1996, 70:3325-9; J. Virol. 1996, 70:7219-23; Antiviral Research 2001, 52:1-17). The validity of these data have been questioned (Laskus et al. 1998, see below). Moreover, PBMC obtained from HCV negative donors were successfully infected using HCV-positive sera, demonstrating that PBMCs are permissive for HCV replication in vitro (J. Gen. Virol. 1995, 76:2485-2491). However, replication of the virus therein was really low. In addition, only RNA was detected. Thus, prior to the present Invention, it remained unclear whether HCV could actively replicate to workable levels in PBMCs.

Using an immunodeficiency (SCID) mouse model that allow long-term survival of human hematopoietic cells Bronowicki et al. (1998) presented strong evidence for persistence of HCV RNA in. PBMCs obtained from HCV positive donors (Hepatology 1998, 28:211-218). The susceptibility of PBMC to HCV infection has been corroborated by in situ hybridization techniques showing both positive and negative polarity RNA strands in circulating and/or bone marrow recruited mononuclear cells. Recent reports have established that HCV is in fact associated to B cells. Based on the model of Epstein-Barr virus another B-cell-tropic virus, that remains latent while the host cell is quiescent but is reactivated and enters a lytic replication phase once the host cell is activated (J. Virol. Methods, 1988, 21:223-227; Annual Rev. Microbiol. 2000, 54:1948). Boisvert et al., (2001) examined the possibility that HCV could replicate in peripheral B cells, but under altered physiological conditions, such as immunosupression or cellular activation. The authors could not detect HCV replication in enriched B cells obtained from HCV positive donors upon cell stimulation with CD40L.

Considering the observations of Laskus et al. (1998) showing the presence of active HCV replication in lymphoid tissue in patients coinfected with HIV (not in non-HIV infected patients), suggesting that co infection of HIV would be required in HCV cell-based assay, and those of Boisvert et al., (2001), it was hypothesized that HCV replication in peripheral blood leukocytes (PBML) requires cell activation (e.g. in the mixture of the T-and B-cell population).

Until now, all studies of HCV replication -have concentrated on documenting the presence of the replicative intermediate (minus-strand) RNA. However, the validity of these reports has been criticized because the presence of viral proteins was not demonstrated. It stands to reason that in order for replication to occur, protein expression is required. Therefore, in order to sustain the observations relating to activated PBMCs, non-structural (NS) HCV proteins were chosen as an indicator of viral replication. The studies presented hereinbelow dearly demonstrated that PBMCs obtained from HCV seropositive donors are able to support at least one complete cycle of viral replication upon activation. For this, a simple method that actively induces virus replication within the infected cell was developed.

Most circulating leukocytes are in a resting state, but remain responsive to mitogenic signal that can induce cell activation. Lymphocyte activation in response to extrinsic signals results in either progression through the cell cycle, or activation of proapoptotic pathway(s) (Cell 1991, 65:921-923; Science 1996, 274:1664-1672). Lymphocyte activation correlates with a strong increase in translation rates and expression of translation” initiation factors (J. Immunol. 1998, 160: 3269-3273). The change in the cellular environment associated with immune activation could induce. HCV protein synthesis and initiate a cascade of events leading to an impaired cell cycle and an enhanced viral replication.

In accordance with the present invention, the activation of PBMCs (or PBLs) is achieved using at least one mitogenic (or activating agent). In one particular embodiment, the activating agent is a mixture of antigen-nonspecific T and/or B cell activators (Anti-CD3 antibody, phytohemagglutinin (PHA), CD40L, Staphylococcus aureus crown I (SAC), IL2 and IL4). Of course, it will be realized that other T and B cell activating agents exist and are well-known in the art. Such agents could be used in the methods and culture systems of the present invention. In one particular embodiment, Ag-specific T and/or B cell activating agents could also be used. It will also be understood that the present invention provides assays which can be used to identify further activating agents, mixtures thereof or other nutrients which can further activate the HCV-producing cells of the present invention and/or promote a longer survival thereof in culture.

HCV non-structural proteins (NS3 and NS5) were detected by Western blot analysis. Virus-like particles could be detected within the infected cells by electron microscopy demonstrating that viral proteins are assembling. Viral particles could be isolated from the PBMCs supernatant. The presence of virus was evidenced from Western blot (anti-Core) analysis and genomic RNA detection by real time RT-PCR, this observation shows that upon assembly, viral particles were actively being liberated to the supematant.

Moreover, using a co-culture method it was demonstrated that the HCV particles produced in PBMC could infect “other cells. Non-limiting examples thereof include liver cells such as Huh-7, Daudi (B-cell) ,MT4 (T-cell) cell lines, naive PBLs and thus B and T cell lines as well as primary lymphocytes. Thus, not only can HCV replicate, and assemble in the tissue culture system of the present invention, it can also infect other cells. Infection was monitored by detection of viral RNA (real time RT-PCR). The results generated by these experiments will have a significant Impact on the testing of anti-HCV agents. Of course, it also serves as a proof of principle that PBMC are able to sustain HCV infection and generate infective HCV. Moreover these data strongly suggest that both the serum and PBMCs obtained from HCV positive donors can be used as a source of infectious virus to infect naive cells such as monocyte and/or monocyte-derived dendritic cells (DCs). Therefore, the instant invention which enables the infection of cells with HCV is by itself a significant achievement.

A Novel Tool for Developing a HCV Vaccine

Adoptive transfer of donor-derived virus-specific T cells generated in cultures with antigen-bearing autologous monocyte-derived dendritic cells (DCs) has attracted considerable attention as a promising tool to generate a strong immune response (Int. J. Cancer. 2001, 94:459-73; Exp. Hematol. 2001, 29:1247-55; Trends Mol. Med. 2001, 7:388-94). This technique has not only proved useful as an alternative anti-cancer strategy but also as a novel anti-virus therapy. For example, when DCs were pulsed with human cytomegalovirus virus (HCMV) antigen and cocultured with autologous peripheral blood lymphocytes from HCMV-seropositive individuals, there was an increase in the numbers of cytolytic T cells. This technique was used to enhance immunity in HCMV-seropositive transplant patients (Blood. 2000, 97: 994-1000).

Now having developed a technology to infect cells with HCV, it becomes possible to adapt the dendritic cells (DCs) technology mentioned above, to generate T-cell responses to HCV. Advantages for using DCs for this purpose include: i) they are considered the most potent of the antigen-presenting cells (APCs) (Blood. 1997, 90:3245-3287; Nature. 1998, 392:245-252); ii) their role in resistance against experimental malignancies and infections is well documented (J. Immunol 1998, 161:2094-2098; J. Virol. 1998, 72:3812-3818); iii) DCs can be easily generated from bone marrow, cord blood, and peripheral blood; iv) DCs have the unique ability to process exogenously supplied antigen efficiently and present peptides on both class 1 and class 2 HLA molecules along with an array of costimulatory molecules (Nature. 1998, 392:245252; Nature. 1999, 398:77-80). The presentation of both helper and CTL-defined epitopes suggests that both CD4+ and CD8+ HCV-specific T cells will be generated. This will allow both the generation of cytolytic effector function and the potential for re-establishment of longer-term immune memory, which may be Important in preventing subsequent viral reactivation; vi) The lack of an absolute knowledge of the presented peptides means that this technique can be used for patients of any HLA type and will trigger T-cell reactivity to undefined immunogenic determinants, thereby allowing a greater potential for augmentation of a broader T-cell response. It is thus expected that this will reduce the possibility that selective pressure will be applied to HCV in vivo. Based on the foregoing, it is predicted that the approach described herein (together with possible adaptations by a person of ordinary skill using the knowledge in the art) will contribute significantly to the design of a vaccine therapy towards HCV infection.

The present” invention is illustrated in further detail by the following non-limiting examples.

### EXAMPLE 1

### Hepatitis C Virus Replication in Peripheral Blood Lymphocytes from Infected Donors

There is considerable evidence that hepatitis C virus (HCV) resides in an extrahepatic reservoir. Although peripheral blood lymphocytes (PBLs) have been suspected of harboring HCV, virus production was not achieved in these cells despite many attempts.

Here, we show that PBLs from HCV positive, injection drug users, harbor the virus and support viral replication. HCV replication was activated by ex vivo cell stimulation, with the use of a mixture of T and B cell activators. The presence of viral positive and negative RNA strands and HCV proteins is documented. Virus particles were isolated from cell supematant and analyzed by density gradients centrifugation. Virus structural proteins and viral RNA could be readily detected in the supematant of activated PBLs by Western blotting and real time RT-PCR, respectively. Virus particles contain de novo synthesized genomic RNA and structural proteins as shown by metabolic labeling with ³²P-orthophosphate and ³S-labeled aminoacids. Finally, HCV particles, released from cells, are infectious as demonstrated by co-culturing. Studies using this novel HCV replication system should contribute to the understanding of the virus life cycle, host-virus relationship, pathogenesis and importantly to the discovery and validation of new anti-HCV agents.

Hepatitis C virus (HCV) is a significant etiologic agent of chronic liver disease (1). It is estimated that more than 170 million people world-wide are seropositive. About 85% of primary infections become chronic, and ˜20% of patients with chronic HCV develop serious complications, such as liver cirrhosis, end-stage liver disease, hepatocellular carcinoma, and death due to liver failure (2). To date, there is no vaccine against HCV and the most effective therapy is treatment with peginterferon in combination with ribavirin (3, 4). The search and validation of novel HCV drugs is severely hampered by the lack of a robust cellular system that supports virus replication. These facts cast HCV as a human pathogen of extreme medical significance.

HCV is an enveloped RNA virus of the Flaviviridae family, classified within the Hepacivirus genus. It contains a 5′uncapped positive strand RNA genome of 9.4 kb, that possesses two overlapping open reading frames: one is translated into a single polyprotein of 3010. aminoacids, while the other yields a 17 kDa protein (5×7). The viral polyprotein is processed to generate at least 10 different structural and nonstructural proteins (5, 6). The genome of HCV is highly heterogeneous and the virus circulates as quasispecies in a single infected individual (8). HCV is primarily hepatotropic, but it has also been implicated in lymphoproliferative diseases such as mixed cryoglobulinaemia, B-cell non-Hodgkin's lymphoma, and Sjogren's syndrome (9). The case for HCV replication in PBLs is suggested by the following observations: a) PBLs from HCV positive donors are capable of transmitting viral infection when inoculated into chimpanzees (10), and b) HCV minus-strand RNA can be “detected in PBLs from HCV carriers upon injection into SCID mice (11). However, despite the growing evidence that supports HCV entry into PBLs, viral RNA synthesis is still a matter of debate and virus replication in PBLs has not been demonstrated (9, 12). Detection of HCV genomic sequences (plus-strand) and replicative intermediates (minus-strand) in PBLs from chronically infected donors (13-16) or infected chimpanzees has been reported (17, 18). But, the presence of viral proteins or virus particles has never been documented. To examine HCV extrahepatic replication, we used PBLs from seventy-eight HCV positive, HIV-negative, injection drug users (IDUs; all obtained with written consent; table S1 detailing the available information on the participants is included in the on-line supplement). PBLs from the IDUs were treated with a mixture of T and B cell activators to show replication of HCV and infectivity of the de novo produced virus. The rationale behind the selection of IDUs as a source of PBLs is addressed below.

HCV RNA and Proteins are Produced de novo in Activated PBLs.

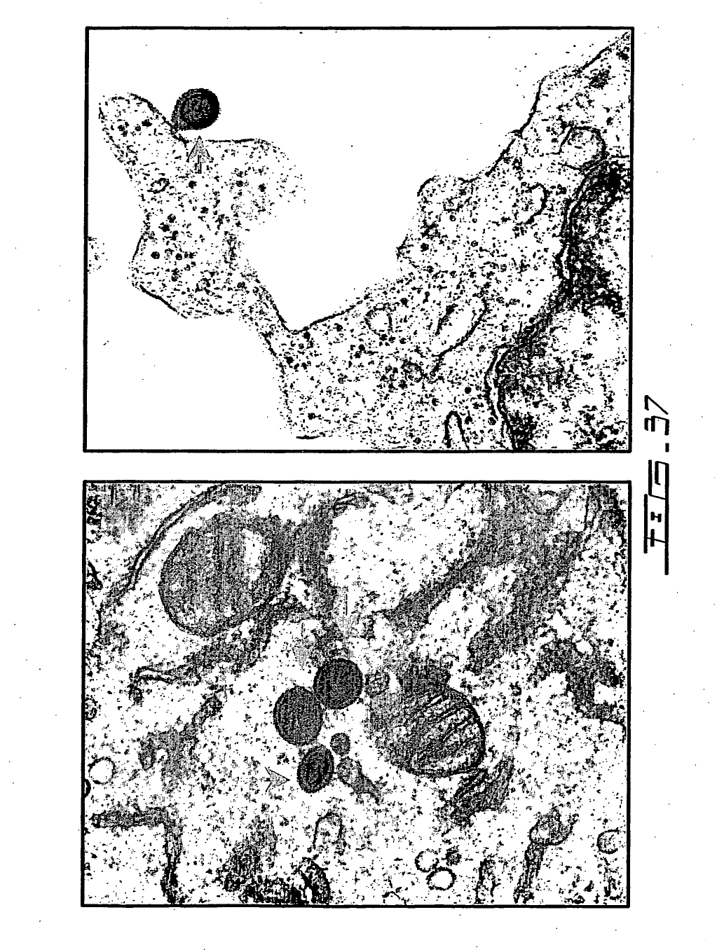

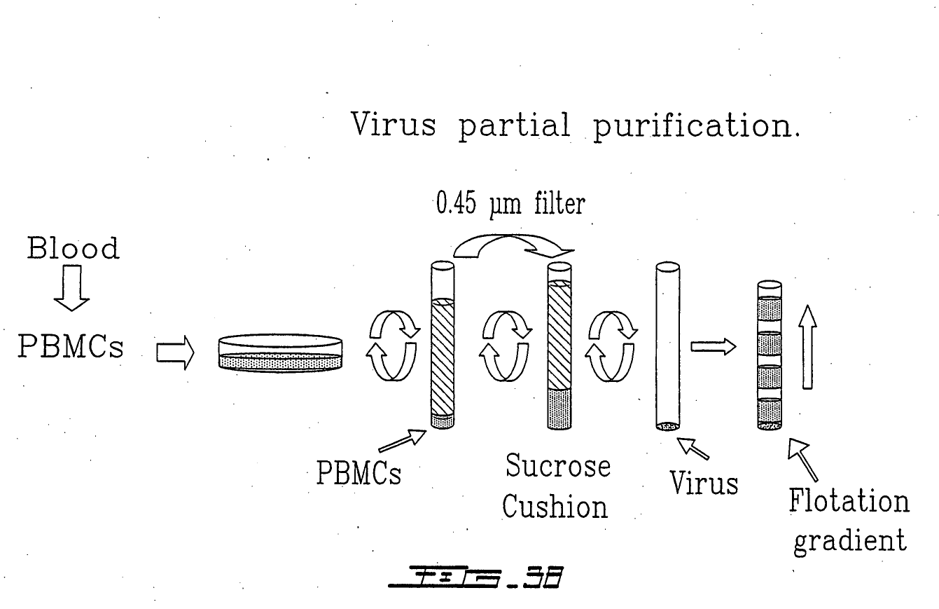

Viral RNA was detected in non-stimulated and stimulated PBLs from a HCV positive donor by nested RT-PCR (FIG. 23A). Viral RNA was not detected in HCV negative donors or in negative controls (FIG. 23A; Note that nested RT-PCR is neither strand specific nor quantitative). These results confirm early evidence showing that PBLs harbor HCV RNA (12-16). To obtain quantitative results, total RNA extracted from activated cells was subjected to a strand specific real time RT-PCR analysis to demonstrate the presence of HCV (−) RNA strand (FIG. 23B). The kinetics of HCV RNA induction was similar in activated PBLs from two carriers, MLL-038 and MLL-039 (FIGS. 23B). The amount of (−) strand RNA increases slightly, but significantly, early (1 day) upon cell activation then decreases at later times (1-3 days), but increases again afterwards (5-7 days) (FIG. 23B). Although these kinetics are not readily explained, the presence of HCV (−) RNA strand supports the notion of virus replication in PBLs. HCV life cycle is cytoplasmic (5), therefore, to show that RNA synthesis occurs in the cytoplasm, bromo-substituted uridine (BrU) together with actinomycin D (ActD) was added to stimulated PBLs (19). Incorporated BrU was detected by immunofluorescence using antibodies to 5′-bromodeoxyuridine (19). Cytoplasmic RNA synthesis was detected in activated HCV positive PBLs from two HCV positive donors (FIG. 23C and 28). In contrast, no incorporation of BrU was detected in ActD treated PBLs from a HCV negative donor (FIG. 23D). In the absence of ActD, strong incorporation of BrU in newly synthesized RNA was detected in the nucleus (FIGS. 23C and D). Taken together, our data clearly show that HCV RNA synthesis occurs in activated PBLs from IDUs.

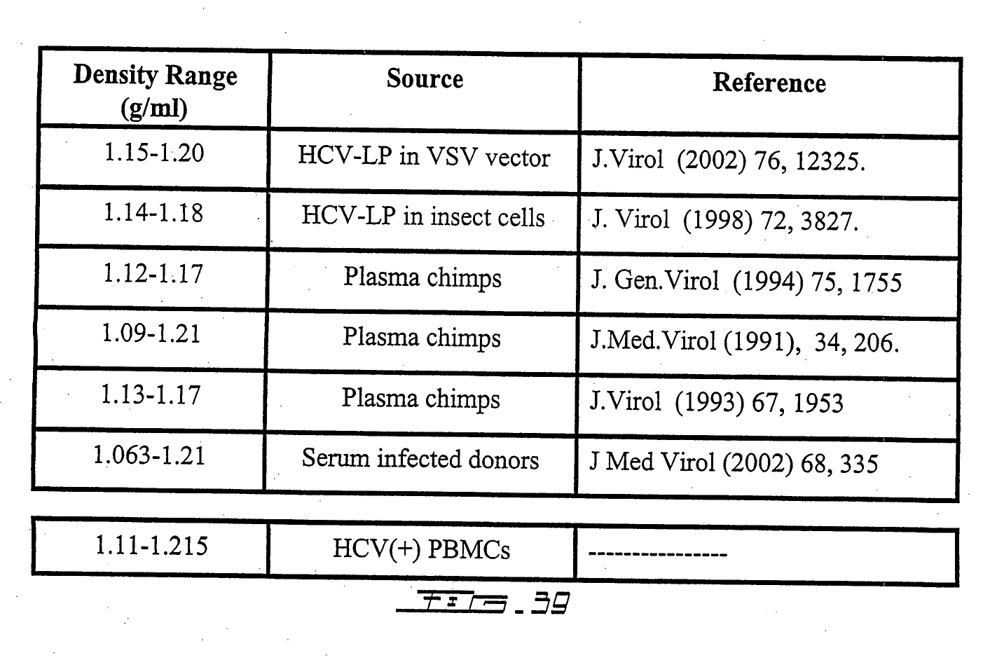

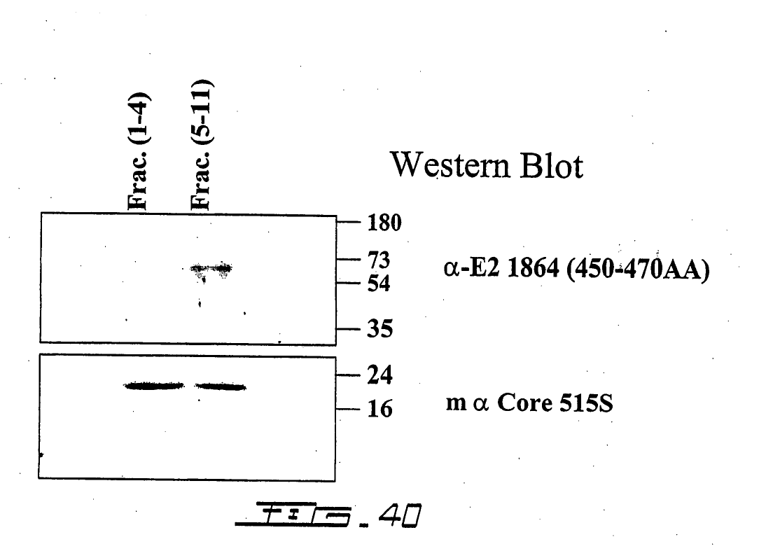

Next, we wished to document HCV-directed translation in PBLs. Upon mitogen stimulation of HCV positive PBLs, NS3 and NS5B proteins were readily detected by Western blotting using several different antibodies (FIGS. 24A-C). The quantity and kinetics of NS3 appearance was dependent on the particular procedure of stimulation (FIGS. 24D and E) and the HCV carrier (FIGS. 24F-H). This suggests that the kinetics of HCV protein production in stimulated PBLs is modulated by host factors. To show that the appearance of the proteins, which interact with the NS3 and NS5B antibodies, is dependent on HCV replication, we used siRNA against the core protein coding sequence (FIGS. 24I-K). NS3 and NS5B levels decreased drastically following electroporation of the Core-siRNA in a dosedependent manner when compared a to a non-specific unrelated RNA (inverted 4E-T-siRNA; see Materials and Methods, below) (FIG. 24I). siRNA silencing resulted from a decrease of HCV RNA, as compared a to a non-specific RNA, as demonstrated by real-time PCR quantification (FIGS. 24J, K).

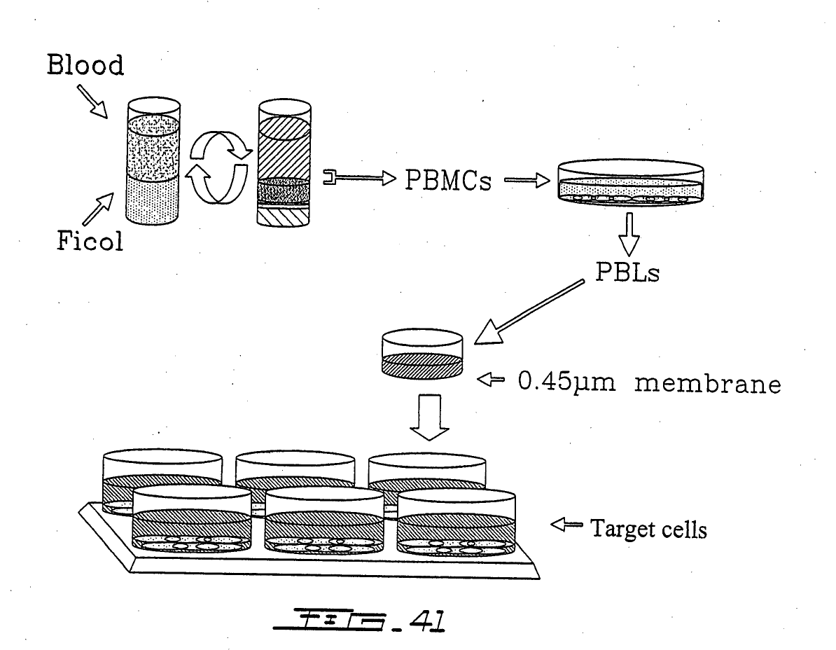

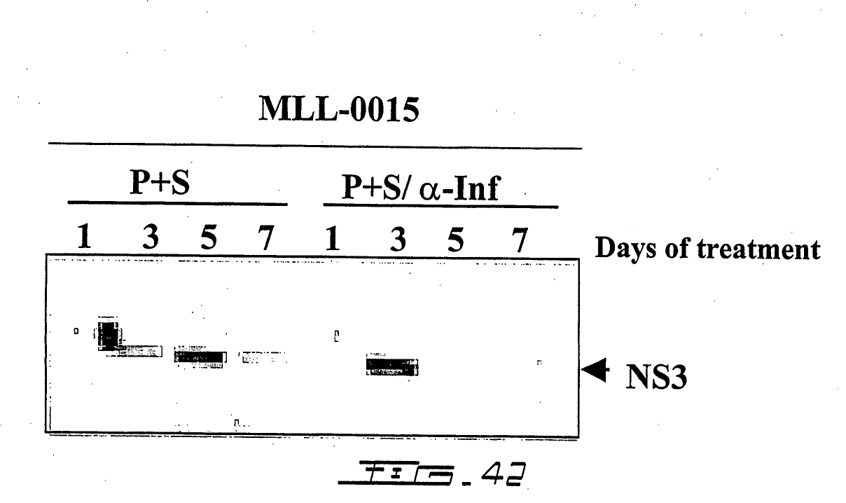

The presence of core protein in the cytoplasm of activated HCV positive PBLs was further confirmed by indirect immunofluorescence (FIG. 25). Based on surveying 10 fields, we estimate that 1 to 3% of the cells expressed high levels of HCV core protein. Taken together, the data demonstrate that translation of the HCV (+) strand RNA (FIGS. 24 and 25 and transcription of the (−) strand RNA (FIG. 23) occur in activated PBLS.

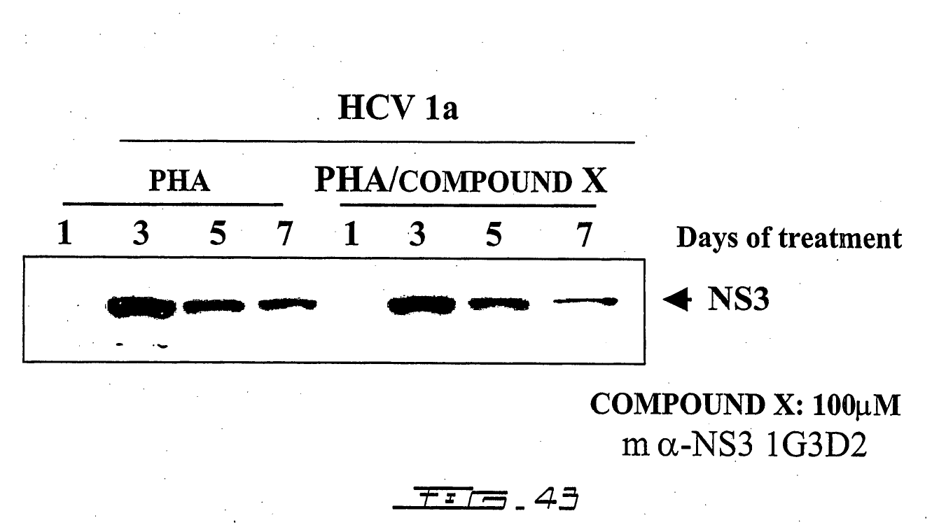

To examine whether HCV particles are produced and released into the culture medium, the supematant from PBLs was harvested and sedimented by centrifugation through a 20% sucrose cushion. The presence of HCV particles was demonstrated by Western blotting with an anti-core monoclonal antibody, MAB225P (FIG. 26A). Similar results were obtained when other anti-core antibodies (monoclonal 515S (20) and polyclonal RR8) were used (data not shown). Viral RNA co-sedimented with the HCV core protein as demonstrated by nested. RT-PCR (FIG. 26B). PBLs were stimulated by methods B, P and PS (detailed in Materials and Methods) and genomic RNA isolated from the cell supematant was quantified by real time RT-PCR (FIG. 26C). Consistent with the protein data shown above, the amount of viral RNA in the cell supernatant varied among the different stimulation procedures (FIG. 26C). To further support the evidence for virus production, particles were examined following metabolic labeling with ³⁵S-methionine/cysteine (FIGS. 26D-G). Particles were sedimented through a 20% sucrose cushion, resuspended and floated on Optiprep™ density gradients (21) (FIG. 26D). The sedimentation range of the labeled particles (1.13-1.215 g/ml) was similar to that reported by others (22-28). HCV-E2 protein was present in the particles as determined by Western blotting using monoclonal anti-E2 1864 (FIG. 26E). The absolute quantity of HCV (+) strand RNA present in each faction was determined by real-time RT-PCR (FIG. 26F). The HCV genomic RNA and E2 co-sedimented through the density gradient (FIG. 26F). Interestingly, Western blotting” revealed that the HCV core protein sedimented” throughout the gradient (data not shown). To further examine this behavior fractions 14 and 5-11 from the gradient were pooled and the presence of HCV E2 and core proteins was determined. The high (H) density complexes (1.111 to 1.215 g/ml) contained E2 and core protein and are likely to represent viral particles, while the low (L) density complexes (1.006 to 1.1 g/ml) contained only core (FIG. 26G). The biological significance of this observation is not immediately clear. However, it was suggested earlier that different types of particles are found in serum from chronically infected individuals (23, 29), and in the supernatant of cells expressing the full length HCV RNA (21). RNA and proteins were isolated following metabolic labeling with 35S methionine/cysteine or 32P-orthophosphate (the latter in the presence of ActD) to determine whether the viral proteins and genomic RNA Isolated from the different fractions was synthesized de novo. Supematant was collected after labeling (FIG. 26H). Significantly, labeled RNA and proteins co-sedimented through the density gradient (FIG. 26H). Thus, the results show that virus particles containing de novo synthesized proteins and genomic RNA were released to the supernatant.

HCV Particles Released from HCV Positive PBLs are Infectious.

It was highly pertinent to examine whether the HCV particles released from stimulated PBLs are infectious. As it is impossible to estimate the real ratio of infectious to non-infectious virus particles produced by activated PBLs, a co-culture strategy, in which two different cell types in two chambers are separated by a 0.45 μm polyethylene” terephthalate track-etched membrane,” was used (FIG. 27A). The HTLV-1transformed T cell line, MT4 was chosen as the target cell of infection (30-33). Total RNA was extracted from infected cells and the quantity of HCV RNA was determined. Strikingly, viral RNA (average of 1600 copies/μg of total RNA; as determined by real-time RT-PCR, data not shown) and NS3 protein were detected in MT4, upon co-culture with activated PBLs (FIG. 27B), demonstrating that the released viral particles are infectious and that cell-to-cell contact is not required for infection. No viral proteins were detected in MT4 cells when co-cultured with PBLs from two HCV negative donors (FIG. 27B).

In conclusion, we demonstrated that HCV replication occurs in PBLs. Without being limited to a particular theory, our success in showing replication, while earlier studies failed, can be attributed to two important factors: activation of the PBLs and the use of IDU donors. IDUs were selected because they experience a long-term altered immune response (34-36) and HCV replication in PBLs has been associated with induced immunodeficiencies (37-39). Drugs have a variety of effects on the immune system including suppressed cell-mediated immunity (34-36). This is reflected in a depressed level of T-dependent antibody production by B lymphocytes and in an alteration of T lymphocyte function. The clinical consequences of this suppression include an increase in the incidence of viral infections such as HIV and HCV (4042). Thus, our observations support the notion that immunosupression in combination with cell activation act as “cofactors” in HCV, pathogenesis. Studies including HCV infected individuals who are not IDUs and non-IDU immuno-suppressed individuals are required to support this hypothesis.

It is most probable that HCV enters lymphocytes during the primary infection and remains latent in resting cells. Viral latency is well documented for Epstein-Barr virus (EBV), which remains dormant in quiescent host B-cells, but enters a lytic replication phase once the cell is activated (43, 44). Interestingly, EBV can also infect T cells (45, 46). Therefore, a number of intriguing parallels can be drawn between the HCV and EBV life cycles. It is conceivable that like in EBV infection, T cell immunity plays a critical role in limiting the number of HCV infected PBLs and that during a sustained immunodeficiency state, such as that manifested in IDUs, clonal proliferation of virus infected cells will be favored. Most importantly, in this report we describe a simple cell-based system that supports robust HCV replication. The implications of these findings are paramount for several reasons. First, they dearly implicate PBLs in HCV pathogenesis. Second, they provide a model that should be useful in the quest to gain understanding of the HCV life cycle, host-virus relationship, viral infectivity and in the discovery and validation of novel anti-HCV agents.

### EXAMPLE 2

### Materials and Methods

Antibodies. A number of antibodies can be used, including NS3 polyclonal antibody, monoclonal anti-NS5B and monoclonal anti-NS3. More specifically, monoclonal anti-NS3 antibody, 1G3D2 and polyclonal anti-NS3, K135 were from “Dr. D. Lamarre (Boehringer Ingelheim Canada Ltd). Monoclonal anti-E2 1864 (450470AA), monoclonal anti-5B 10 (IFA), monoclonal anti-Core 515S (2040AA), and Core rabbit anti-serum RR8 were developed in The Tokyo Metropolitan Institute of Medical Science. Monoclonal anti-Core (Cat.No.: MAB255P; Lot:hcv-core-24) was purchased from Maine Biotechnology services, Inc. Monoclonal anti-human β-Actin (ab205) was purchased from Abcam Limited. Monoclonal anti-human PActin (clone AC-15) was purchased from Sigma-Aldrich CO. Anti-Bromodeoxyuridine” monoclonal antibody-Alexa fluor 488 conjugated, and goat anti-rabbit. Alexa fluor 594 conjugated were purchased from Molecular Probes, Inc.

Blood Donors and lymphocyte purification. Participants were recruited through the drug addiction unit of the Saint-Luc Hospital of the Centre Hospitalier de l'Université Montreal (CHUM) and the Saint-Luc Cohort study. Characteristics of the donors can be found at Table 1. Donors provided a written informed consent approved by the CHUM Review Board before having their blood drawn. Individuals from both sexes (87% males) were enrolled in this study between 2001 and 2003. Their mean age was 42.1 years (sd±8.8) and the average time since their first injection was 16.5 years (sd±9.6). 80% of the donors reported injecting drugs during the 6 month period before blood was withdrawn for this study. Cocaine and opiates were the most frequently used drugs, with 77% and 34.6% use, respectively. All HCV positive donors tested positive in a serological screen for HCV antibodies performed in the laboratory of microbiology at Saint-Luc Hospital of the CHUM using two Enzyme Linked Immunosorbent Assays (ELISA, AxSym and Cobas). Presence of HCV was confirmed by HCV-RNA detection when ELISA data were discordant. All participants recruited for this study were HIV-1 and HIV-2 negative. Serological screening for HIV antibodies was performed in the microbiology laboratory at Saint-Luc Hospital, CHUM, with an enzyme-inked immunosorbent assay (ELISA). Similar procedures were used to verify the HCV negative donors. HCV negative donors (six) were recruited from the different participating laboratories as well as from the support staff responsible for the St. Luc Cohort. Peripheral blood (20 ml) was collected from HCV positive IDU or HCV negative donors into EDTA-containing Vacutainer tubes (Becton Dickinson). Polymorphonuclear leukocytes and red blood cells were separated by centrifugation over a density gradient (Lymphocyte separation medium, cellgro®). Monocytes were then removed by plastic adherence under serum free conditions as described in The Current protocols of Immunology. When required, cells were frozen in 10% DMSO containing FCS and stored at −80° C. prior to monocyte separation. Total PBLs were cultured in 24-well plates at 1×10⁶ cells per ml in RPMI 1640 supplemented with 10% heat-inactivated FCS and antibiotics.

PBLs stimulation. Mitogens were added to the media (RPMI 1640, 10% FBS, and antibiotics) upon starting the culture and maintained throughout the experiment. The protocols used for PBCLs stimulation were as follows: Method A, PBLs were grown in the presence of irradiated L4.5 cells (murine fibroblasts expressing the CD40 ligand, CD154) as described (49). Method B, 1 μg/ml of anti-CD3 and 200 U/ml of IL-2 (Sigma-Aldrich CO) were added. Method P, 3 μg/ml phytohemagglutinin (PHA, Sigma-Aldrich CO), and 200 U/ml IL-2 were used. Method PS, 1:10⁴ vol/vol of Staphylococcus aureus Cowan fixed cells (SAC, Calbiochem) in combination with phytohemagglutinin and 200 U/ml IL-2 were added to the media. Method S, 10⁴ volvol of SAC and 200 U/ml of ILK (Sigma-Aldrich CO) were used. Cell activation was verified by flow cytometry. Cells were rinsed twice with 1 ml cold phosphate buffered saline (PBS: 137 mM NaCl, 2.7 mM KCl, 4.3 mM Na₂HPO₄, 1.4 mM KH₂PO₄, pH 7.4) and fixed in 80% ethanol/PBS for 30 min at 4° C. PBS (2 volumes) was added and cells were pelleted by centrifugation. Cells were rinsed twice with 2 ml PBS and then resuspended in 0.5 mL PBS containing 0.2 μg/ml RNase A and incubated for 40 min at 37° C. Propidium iodide was added to a final concentration of 1.2 μg/ml and samples were analyzed by flow cytometry using a single laser FACS instrument (Becton-Dickinson) combined with the CellQuest™ software.

RNA puriflcation. Total RNA was extracted from cells using Trizol™ (Invitrogen) according to the manufacturer's protocol. Yeast tRNA (1 mg/ml) was added as a carrier. RNA was resuspended in nuclease-free water (Sigma-Aldrich CO). Total RNA was quantified by Phosphoimager™ (STORM system, Molecular Dynamics) using the RiboGreen™ RNA Quantification Kit (Molecular Probes, Inc).

Nested RT-PCR. HCV-RNA was detected in cells by a reverse transcription-polymerase-chain reaction (one step RT-PCR reaction, 45 cydes, Qiagen) against the highly conserved 5′ untranslated region (sense primer from nucleotide 13 to 38 and the anti-sense primer from nucleotide 383 to 359) of the HCV genome (strain H77 pCV-H77C, EMBL:AF011751, MEDLINE: 97385173) followed by a second round of amplification, nested PCR (45 cydes, sense primer from nucleotide 59 to 82 and the anti-sense primer from nucleotide 307 to 285, strain H77 pCV-H77C) using Taq DNA polymerase (MBI Fermentas). β0Actin was amplified (30 cycles) using the sense primer 5′-GTGGGGCGCCCCAGGCACCA-3′ and antisense primer 5′-GTCCTTAATGTCACGCACGATTTC-3′.

### EXAMPLE 3

### Assessement of the Sensitivity/Resistance Profile of HCV Strain or use of the Assay of the Present Invention to Screen and Select for Anti-HCV Compounds

In the absence of an effective antiviral or vaccination strategy against HCV, the single drug that is often used to treat chronic HCV infection is alpha-IFN, a naturally occurring glycoprotein that has antiviral and immunomodulatory properties. It continues to be the only known drug to induce sustained HCV clearance and cause an improvement in liver histology. However, IFN-monotherapy is limited by adverse side effects. Furthermore, a sustained virological iesponse (SVR) is achieved in only 15% of patients. The combination of the orally active synthetic guanosine analogue ribavirin with IFN-2b has proved to be more effective than IFN monotherapy, yielding an SVR in 35-40% of patients. Ribavirin action is thought to reside, at least in part, in its ability to inhibit inosine monophosphate dehydrogenase (IMPDH), an enzyme that catalyses a rate-limiting step in GTP biosynthesis. This leads to a decreased intracellular pool of GTP levels, and therefore indirectly suppresses the synthesis of viral RNA. The antiviral activity of ribavirin might also be related to its ability to inhibit the HCV NS5B polymerase directly.

However, despite the improved efficacy of the combination therapy of IFN- and ribavirin, most patients still fail to achieve an SVR to the treatment. Furtherrnore, side effects have also been described for the combination therapy.

FIG. 42 shows that the sensitivity/resistance phenotype of HCV to the known anti-HCV compound α-IFN can be determined by the assay of the present invention. As shown in FIG. 42 Alpha-INF has an effect, it is able to reduce virus replication between day 3 and day 5. Without the drug we can even see replication on day 7 post stimulation. This is what would be expected for an INF sensitive individual.

Of course, the different candidate anti-HCV compounds could be screened using the assays of the present invention. In addition, the present invention provides the means to assess the resistance/phenotype profile of patients' strains of HCV toward a particular anti-HCV compound or candidate or pool thereof.

Non-limiting examples of compounds that could be used in such phenotype determination are listed In Tables 1 and 2.

TABLE 1
Selected IFN-based therapies for the treatment of HCV infection

| Drug name | Company | Web site | Clinical phase |
|---|---|---|---|
| Monotherapy | | | |
| Intron A (IFN-α2b, recombinant) | Schering-Plough | http://www.sch-plough.com | FDA approval, 1995 |
| PEG-INTRON (PEGylated IFN-α2b) | Schering-Piough | http://www.sch-plough.com | FDA approval, 2001 |
| Roferon A (IFN-α2b, recombinant) | Roche | http://www.roche.com | FDA approval, 1996 |
| Pegasys (PEGylated IFN-α2b) | Roche | http://www.roche.com | FDA approval, 2001 |
| Infergen A (IFN alfacon-1) | InterMune Pharmaceuticals | http://www.intermune.com | FDA approval, 1997 |
| Wellferon (lymoblastoid IFN-αn1) | GlaxoSmithKline | http://www.corp.gsk.com | FDA approval, 1999 |
| Omniferon (natural IFN-α) | Viragen (Scotland) | http://www.viragen.com | Phase II |
| Omega IFN(IFN-ω) | BioMedicines | http://www.biomedicinesinc.com | Phase II |
| Albuferon-α (albumin-IFN-α2b) | Human Genome Sciences | http://www.hgsi.com | Phase I |
| Rebif (IFN-β1a) | Serono | http://www.serono.com | Preclinical* |
| Combination Therapies | | | |
| Rebetron (Intron A and ribavirin) | Schering-Plough | http://www.sch-plough.com | FDA approval, 1998 |
| PEG-INTRON and ribavirin | Schering-Plough | http://www.sch-plough.com | FDA approval, 2001 |
| Pegasys and ribavirin | Roche | http://www.roche.com | FDA application |
| | | | submitted |
| Intron A and Zadaxin (α1-thymosin) | RegeneRx Biopharmaceuticals/ | http://www.regenerx.com | Phase III |
| | SciClone Pharmaceuticals | http://www.sciclone.com | |
| Pegasys and Ceplene | Maxim Pharmaceuticals | http://www.maxim.com | Phase III |
| IFN-β and EMZ701 | Transition Therapeutics | http://www.transitiontherapeutics.com | Preclinical |

*FDA approval for the treatment of relapsing forms of multiple sclerosis.
HCV, hepatitis C virus;
IFN, interferon;
PEG, polyethylene glycol

TABLE 2
A sample of the drug pipeline for hepatitis C and related treatments

| Target/ | | Mechanism/ | | Clinical |
|---|---|---|---|---|
| indication | Drug name | drug category | Company | Phase |
| IRES | ISIS 14803 | Antisense | ISIS Pharmaceuticals/ | Phase II |
| | | | Elan Corporation | |
| | Heptazyme | Ribozyme | Ribozyme Pharmaceuticals | Phase II |
| NS3 | BILN-2061 | Serine-protease inhibitor | Boehringer Ingelheim | Phase II |
| | VX-950/LY-57031 0 | Serine-protease inhibitor | Vertex Pharmaceuticals/Lilly | Preclinical |
| NS5B | JTK-003 | RdRp inhibitor | Japan Tobacco | Phase I/II |
| E1 | Not known; a recombinant E1 | Therapeutic vaccine | Innogenetics | Phase IIa |
| E2 | XTL-002 | Monoclonal antibody | XTL Biopharmaceuticals | Phase Ib |
| IMPDH | VX-497 | IMPDH inhibitor | Vertex Pharmaceuticals | Phase II |
| | Levovirin | IMPDH inhibitor | Ribapharm | Phase I |
| | Viramidine | IMPDH inhibitor | Ribapharm | Phase I |
| Liver fibrosis | Actimmune (IFN-γ) | Antifibrotic | InterMune Pharmaceuticals | Phase II |
| | IP-501 | Antifibrotic | Intemeuron Pharmaceuticals | Phase III |
| Liver apoptosis | IDN-6556 | Caspase inhibitor | Idun Pharmaceuticals | Phase II |
| HCC | T67 | β-tubulin inhibitor | Tularik | Phase III |
| HCV re-infection | Civacir | HCV IgG | Nabi Pharmaceuticals | Phase I/II |
| | CellCept (Mycophenolate mofetil) | Immunosuppressant | Roche Holdings | Preclinical |
| Target unknown | Ceplene (histamine dihydrochloride) | Immune modulator | Maxim Pharmaceuticals | Phase II |
| | Zadazin (thymosin α-1) | Immune modulator | SciClone Pharmaceuticals | Phase III |
| | Symmetrel (amantadine hydrochloride) | Broad antiviral agent | Endo Laboratories | Phase IV |

*Suspended pending toxicology investigation.
HCC, hepatocellular carcinoma;
HCV, hepatitis C virus;
IFN, interteron;
IgG, immunoglobulin G;
IMPDH, inosine monophosphate dehydrogenase;
IRES, internal ribosome-entry site;
NS, non-structural protein;
RdRp, RNA-dependent RNA polymerase.

An example of the assay of the present invention to screen a candidate compound for anti-HCV acting is shown in FIG. 43.

FIG. 43 shows that compound X reduces by about 2-3 fold the expression of NS3 such as assay of the present. invention (which could be automated to permit high throughput screening for example) is this validated for drug screening. Western Blots. Proteins extracts were prepared by sonification in RIPA buffer (150 mM NaCl, 1% NP40, 0.5% DOC, 0.1% SDS, 50 mM Tris-HCl pH -7.5) and quantified (BSA assay, BioRad). Proteins (10 μg of extracts from PBLs or 5 μg of extract from Huh7 cells, stably expressing the HCV replicon (47)) were resolved on SDS-10% polyacrylamide gels (PAGE) and transferred to 0.2 tlm Protran nitrocellulose membrane (Schleider and Schuell) for 1 h at 100V. The membrane was blocked with PBS containing.0. 5% Tween-20 (PBS-T) and 5% nonfat dry milk. Blots were then incubated with the primary antibody for 2 h at room temperature, washed 3 times with PBS-T and incubated for 1 h with a horse radish peroxidase (HRP) conjugated secondary antibody. Blots were visualized using an enhanced luminol reagent (ECL; PerkinElmer Life Sciences Inc).

Radio labeling and gradient purification of virus particles. A total of 1×10⁶ activated . PBLs were first preincubated in Mrethionine or phosphate-free RPMI for 30 min, and then incubated for 12 h in the same media supplemented with [³⁵S] protein labeling mix (1175 Ci/mmol) or carrier-free inorganic. ³²P (500 μCi/ml, H₃PO₄, ICN Biomedicals, INC), the latter in presence of ActD (5 μg/ml). Supematant was collected, cells and cellular debris was removed by low-speed centrifugation at 1600×g for 15 min at 4° C., followed by filtration with 0.45 μm pore size filter (Fisherbrand, Fisher scientific). Particles were partially purified by ultracentrifugation through a 20% sucrose cushion for a minimum of 6 h at 4° C. (in Beckman L8-55 ultracentrifuge) at 35,000 rpm in a SW-41 rotor. Sediments were resuspended in serum free RPMI and rodixanol (Optiprepm, lnvitrogen) was added to a final concentration of 40% w/v (ρ=1.216). The sample was laid over a 60% wttvol Optiprep™ solution (ρ=1.320 g/ml) and then overlaid with a linear iodixanol gradient (ρ=1.038 to 1.205 g/ml) prepared in RPMI and spun for 20 h at 4° C. in Beckman L8-55 ultracentriftge at 30,000-rpm using a SW41 rotor. Fractions were collected from the top of the tube and RNA was prepared as described above. Half of the final RNA volume was mixed with liquid scintillation cocktail (EcoLitem, ICN Biomedicals) and ³²P radioactivity was counted in a Beckman LS 6500 scintillation counter. Proteins were extracted by directly adding 1OX RIPA buffer to a final concentration of 1× RIPA. 1/100th of the protein extract was mixed with liquid scintillation cocktail and ³⁵S radioactivity. was determined using a Beckman LS 6500 scintillation counter. 1/10 of the protein extract was directly mixed with concentrated Laemmli sample buffer, resolved on a SDS 15%-PAGE, and transferred to 0.2 μm Protran nitrocellulose membrane over night at 30V. The membrane was dried and exposed against Kodak Biomax™ MR film. The remaining protein extract. was concentrated by TCA precipitation (15%-final). Proteins were washed twice with ether, dried and dissolved in a solution containing 3 M urea, 26 mM EDTA (pH 8), and 0.5 μg/ml of RNase A. Samples were mixed with concentrated Laemmli sample buffer, resolved on a SDS 10%. PAGE and transferred to 0.2 μm Protran nitrocellulose membrane for 1 h at 100V. Proteins were detected by Western blotting as described above.

siRNA The target sequence for the siRNA was chosen using the Ambion web-based criteria. The selected RNA oligonucleotides, Core (from nucleotide 371 to nucleotide 391, strain H77 pCV-H77C, EMBL: AF011751, MEDLINE: 97385173) and the unrelated non-specific RNA (inverted sequence for 4E-T from nucleotide 986 to nucleotide 1008; DDBJ/EMBUGenBank database, accession No. AF240775), were synthesized by Dharmacon Research (Lafayette, CO) and handled according to the manufacturer's instructions. Varying amounts (3 μl or 5 μl of a 20 μM solution) of RNA-duplexes were electroporated using a Gene pulser® II electroporator (BioRad), into 1×10⁶ PBLs in 0.5 ml of serum free RPMI. Cells were treated with a pulse of 975 μF and 300 V. Then 0.5 ml of RPMI containing 20% FCS was added and the cells were seeded in a 24-well cell culture dish. Protein and RNA extracts were harvested 48 h after electroporation. Immunoblots were performed as described above using an NS3 rabbit antiserum and monoclonal anti-NS5B. HCV RNA levels were quantified by real-time RT-PCR.

### CONCLUSIONS

The present invention relates among other things to the fact that: (1) HCV has PBMC tropism; (2) HCV can naturally infect blood cells; (3) HCV can replicate in PBMCs and PBLs; (4) HCV replicating In naturally infected PBMCs is infectious; (5) HCV can replicate in extrahepatic tissue; and (6) HCV has a latent phase during PBMC infection, which can be ended by activation.

It is interesting to note that HCV replication is activated upon immune response. Thus, a person of ordinary skill in the art will be able to provide other methods of activation than those disclosed herein (or complementary thereto) to activate HCV replication in PBMCs or PBLCs, without undue experimentation.

The present invention provides the tools to study hepatitis C virus replication in a simple cell culture based system. This simple culturing tool is suitable for the search and validation of novel HCV antiviral drugs and therapies (vaccine). The assays and methods of the present invention enable the performance of screening assays to identify antiviral agents. Of course, the assays, can be highthroughput. Compound libraries can now be used to identify candidate anti-HCV agents. These assays can thus be used to generate lead compounds for pharmaceutical anti-HCV formulations.

The novel replication system of the present invention, in one embodiment, based on PBMCs (or PBLs) is simple, does not require facilities other than those normally used for HIV research, and allows experiments with the complete HCV. Thus, novel drugs and therapies can be screened to target all the different stages of virus replication such as virus entry, cytoplasmic replication (viral (−) and (+) strand synthesis), viral protein synthesis, virus assembly, virus trafficking, and virus release.

TABLE 3
Characteristics of the IDU donors, enrolled between March 2001 and April 2003:

| | | | | | | Opioids excl | |
|---|---|---|---|---|---|---|---|
| | | | IDU | Under | IDU | methadone | Cocaine |
| | Age | | duration | Methadone | (past 6 | (past 6 | (past 6 |
| Participant | (years) | Sex | (years) | treatment | months) | months) | months) |
| SB-1 | 41 | male | 22 | yes | no | no | no |
| SB-2 | 42 | female | 20 | yes | yes | yes | yes |
| SB-4 | 35 | male | 11 | yes | yes | yes | yes |
| SB-5 | 21 | female | 3 | yes | yes | yes | no |
| SB-6 | 32 | male | 1 | yes | yes | yes | yes |
| SB-7 | 45 | male | 18 | yes | no | no | no |
| MLL 001 | 48 | male | 31 | no | yes | yes | yes |
| MLL 002 | 39 | male | 3 | no | yes | no | yes |
| MLL 003 | 38 | male | 10 | no | yes | yes | yes |
| MLL 004 | 47 | male | 32 | no | yes | yes | yes |
| MLL 005 | 38 | male | 21 | no | yes | no | no |
| MLL 006 | 49 | male | 37 | yes | yes | yes | no |
| MLL 007 | 61 | male | 36 | no | yes | no | no |
| MLL 008 | 39 | male | 13 | no | no | no | yes |
| MLL 009 | 23 | male | 5 | no | yes | no | no |
| MLL 010 | 40 | male | 21 | no | no | no | yes |
| MLL 011 | 45 | male | 6 | no | yes | no | yes |
| MLL 012 | 48 | male | 14 | no | yes | yes | yes |
| MLL 013 | 49 | male | 24 | no | no | yes | no |
| MLL 014 | 41 | male | 18 | no | yes | no | yes |
| MLL 015 | 38 | male | 6 | no | yes | yes | yes |
| MLL 016 | 34 | male | 11 | no | no | no | no |
| MLL 018 | 42 | male | 13 | no | yes | no | yes |
| MLL 019 | 51 | male | 10 | no | yes | no | yes |
| MLL 020 | 38 | male | 13 | no | yes | no | yes |
| MLL 021 | 35 | female | 5 | no | no | no | no |
| MLL 022 | 43 | male | 29 | no | yes | no | yes |
| MLL 023 | 52 | male | 20 | no | yes | no | yes |
| MLL 024 | 37 | male | 13 | no | yes | no | yes |
| MLL 025 | 36 | male | 18 | yes | yes | yes | yes |
| MLL 026 | 29 | female | 13 | yes | yes | yes | yes |
| MLL 027 | 52 | male | 11 | no | yes | yes | yes |
| MLL 028 | 45 | male | 6 | no | yes | yes | yes |
| MLL 029 | 42 | male | 6 | no | yes | yes | yes |
| MLL 030 | 43 | male | 10 | no | yes | no | yes |
| MLL 031 | 36 | male | 19 | yes | yes | no | yes |
| MLL 032 | 22 | male | 11 | yes | yes | yes | no |
| MLL 033 | 24 | male | 7 | yes | yes | yes | yes |
| MLL 034 | 52 | male | 26 | no | yes, | no | yes |
| MLL 035 | 61 | male | 36 | no | yes | no | no |
| MLL 036 | 49 | male | 31 | no | yes | no | yes |
| MLL 037 | 57 | male | 36 | no | no | no | no |
| MLL 038 | 27 | male | 11 | no | yes | yes | yes |
| MLL 039 | 42 | female | 17 | yes | yes | yes | no |
| MLL 040 | 53 | male | 40 | no | yes | yes | yes |
| MLL 041 | 34 | male | 11 | no | no | no | yes |
| MLL 042 | 47 | male | 7 | no | yes | no | yes |
| MLL 043 | 42 | female | 23 | no | no | no | no |
| MLL 044 | 30 | male | 11 | no | no | no | yes |
| MLL 045 | 41 | male | 22 | no | yes | no | yes |
| MLL 046 | 43 | male | 21 | no | yes | yes | yes |
| MLL 047 | 41 | male | 18 | no | yes | no | yes |
| MLL 048 | 47 | male | 22 | no | yes | no | yes |
| MLL 049 | 52 | male | 11 | no | no | no | yes |
| MLL 050 | 33 | male | 10 | no | yes | no | yes |
| MLL 051 | 45 | male | 30 | yes | yes | no | yes |
| MLL 052 | 33 | male | 8 | no | yes | no | yes |
| MLL 053 | 43 | female | 12 | no | yes | no | yes |
| MLL 054 | 46 | male | 22 | no | yes | no | yes |
| MLL 055 | 36 | female | 21 | yes | yes | no | yes |
| MLL 056 | 40 | male | 14 | no | no | no | yes |
| MLL 057 | 37 | male | 9 | no | yes | yes | yes |
| MLL 058 | 45 | male | 30 | yes | yes | no | yes |
| MLL 059 | 50 | male | 30 | no | yes | yes | yes |
| MIL 060 | 35 | male | 12 | yes | yes | yes | no |
| MLL 061 | 46 | male | 7 | no | no | no | yes |
| MLL 062 | 48 | male | 11 | yes | yes | yes | yes |
| MLL 063 | 66 | female | 35 | no | yes | no | yes |
| MLL 064 | 38 | male | 3 | no | yes | no | yes |
| MLL 065 | 33 | male | 10 | no | yes | no | yes |
| MIL 066 | 48 | male | 11 | yes | no | no | no |
| MIL 067 | 46 | male | 11 | no | yes | no | yes |
| MLL 068 | 42 | male | 6 | no | yes | no | yes |
| MLL 069 | 42 | male | 23 | no | yes | yes | yes |
| MLL 070 | 44 | male | 11 | no | yes | no | yes |
| MLL 071 | 47 | female | 22 | no | yes | no | yes |
| MLL 072 | 61 | male | 16 | no | yes | no | yes |
| MLL 073 | 37 | male | 9 | yes | no | no | no |

Although the present invention has been dlscribed hereinabove by way of preferred embodiments thereof, it can be modified without departing from the spirit and nature of the subject Invention as defined in the appended claims.

### REFERENCES

1. Journal Of Viral Hepatitis 6, 35-47. (1999).

2. J. H. Hoofnagle, Hepatology26, 15S-20S (1997).

3. J. G. McHutchison, M. W. Fried, Clin Liver Dis 7, 149-61. (2003).

4. S. L. Tan, A. Pause, Y. Shi, N. Sonenberg, Nat Rev Drug Discov 1, 867-81. (2002).

5. R. Bartenschlager, V. Lohmann, J Gen Virol 81,.1631-48. (2000).

6. K. E. Reed, C. M. Rice, Curr. Top. Microbiol. Immunol. 242, 55-84. (2000).

7. Z. Xu et al., EMBO J 20, 3840-8. (2001).

8. J. Gomez, M. Martell, J. Quer, B. Cabot, J. I. Esteban, Journal Of Viral Hepatitis 6, 3-16. (1999).

9. A. L. Zignego, C. Brechot, Journal Of Hepatology 31, 369-76. (1999).

10. J. A. Hellings, J. van der Veen-du Prie, R. Snelting-van Densen, R. Stute, J Virol Methods 10, 321-6. (1985).

11. J. P. Bronowicki et al., Hepatology 28, 211-8. (1998).

12. J. Boisvert et al., Journal Of Infectious Diseases 184, 827-35. (2001).

13. A. L. Zignego et al., Journal Of Hepatology 15, 382-6. (1992).

14. J. T. Wang, J. C. Sheu, J. T. Lin, T. H. Wang, D. S. Chen, Journal Of Hepatology 16, 380-3 (1992).

15. H. M. Muller et al., J Gen Virol 74, 669-76 (1993).

16. J. Bartolome, I. Castillo, J. A. Quiroga, S. Navas, V. Carreno, J Exp Med 178, 17-25 (1993).

17. Y. K. Shimizu et al., J Virol 71, 5769-73 (1997).

18. Y. K. Shimizu et al., Infection 26, 1514 (1998).

19. E. G. Westaway, A. A. Khromykh, J. M. Mackenzie, Virology 258, 108-17. (1999).

20. K. Yasui et al., J Virol 72, 6048-55. (1998).

21. T. Pietschmann et al., J Virol 76, 4008-4021 (2002).

22. H. J. Ezelle, D. Markovic, G. N. Barber, J Virol 76, 12325-34. (2002).

23. W. Pumeechockchai et al., Journal Of Medical Virology 68, 335-42. (2002).

24. T. F. Baumert, S. Ito, D. T. Wong, T. J. Liang, J Virol 72, 3827-36. (1998).

25. D. Bradley et al., Journal Of Medical Virology 34, 206-8. (1991).

26. M. Kaito et al., J Gen Virol 75, 1755-60. (1994).

27. M. Hijikata et al., J Virol 67, 1953-8 (1993).

28. R. J. Carrick, G. G. Schlauder, D. A. Peterson, I. K. Mushahwar, J Virol Methods 39, 279-89. (1992).

29. P. Maillard et al., J Virol 75, 8240-50. (2001).

30. Y. K. Shimizu, A. Iwamoto, M. Hijikata, R. H. Purcell, H. Yoshikura, Proceedings Of The Natlonal Academy Of Sciences Of The United States Of Amerca 89, 5477-81 (1992).

31. T. Mizutani, N. Kato, M. Ikeda, K. Suglyama, K. Shimotohno, Biochemical And Biophysical Research Communications 227, 822-6 (1996).

32. T. Mizutani et al., J Virol 70, 7219-23 (1996).

33. M. Ikeda et al., Journal Of Hepatology 27, 445-54 (1997).

34. M. P. Nair et al., Clin Diagn Lab Immunol 4, 127-32. (1997).

35. T. Pellegrino, B. M. Bayer, J Neuroimmunol 83, 13947. (1998).

36. H. Friedman, C. Newton, T. W. Klein, Clin Microbiol Rev 16, 209-19. (2003).

37. T. Laskus, M. Radkowski, L. F. Wang, H. Vargas, J. Rakela, Am J Gastroenterol 93, 2162-6 (1998).

38. T. Laskus et al., Journal Of Infectious Diseases 181, 442-8 (2000).

39. M. Radkowski, L. F. Wang, H. E. Vargas, J. Rakela, T. Laskus, Hepatology 28, 1110-6(1998).

40. B. Rouveix, Therapie 47, 503-12. (1992). 41. G. C. Baldwin, M. D. Roth, -D. P. Tashkin, J Neuroimmunol 83, 133-8. (1998).

42. M. Resti et al., Clin Infect Dis 35, 236-9. (2002).

43. F. Schwarzmann, M. Jager, N. Prang, H. Wolf, Int J Mol Med 1, 137-42. (1998).

44. F. Schwarzrnann, M. Jager, M. Homef, N. Prang, H. Wolf, Leuk Lymphoma 30, 123-9. (1998).

45. H. Yoshiyama, N. Shim izu, K. Takada, EMBO J 14, 3706-11. (1995).

46. H. Kanegane et al., Leuk Lymphoma 34, 603-7. (1999).

47. V. Lohmann et al., Science 285, 110-3. (1999).

48. D. Moradpour et al., J Biol Chem 277, 593-601. (2002).

49. M. M. Loembe, J. Lamoureux, N. Deslauriers, A. Darveau, R. Delage, Br J Haematol 113, 699-705. (2001).

50. G. Haukenes, A. M. Szilvay, K. A. Brokstad, A. Kanestrom, K. H. Kalland, Biotechniques 22, 308-12. (1997).

1. An in vitro culture system, which is suitable for the replication of hepatitis C virus (HCV), comprising:
HCV-infected cells cultivated in the presence of an HCV-activating composition, said activating composition comprising at least one mitogen; and
a non-infected cell type which is injectable with HCV, whereby said activating composition enables a full replication cycle of said HCV in both the originally infected cells and non-infected cell type.

2. The system of claim 1, wherein said activating composition also comprises a cytokine.

3. The system of claim 1, wherein said activating composition is selected from the group consisting of a) phytohaemagglutinin and IL-2; b) Staphyloccoccus aureus crown 1 (SAC) and IL-4; and c) SAC, IL-2 and IL-4.

4. A tissue culture system for HCV which enables the screening and development of drugs and therapies for virus entry, virus replication, viral protein synthesis, virus assembly, virus trafficking, or virus release.

5. A method of generating a vaccine to HCV comprising a pulsing of monocyte-derived dendritic cells (DCs) with HCV, co-cultured with autologous peripheral blood lymphocytes from a HCV-seropositive individual.

6. A method of activating the replication of HCV in PBMCs or PBLs comprising obtention of same from a HCV-infection patient and activating the replication of HCV by incubating the PBMCs or PBLs with an activation-inducing amount of at least one mitogen.

7. A co-culturing system for replicating HCV in vitro which comprises co-culturing PBMCs (or PBLs) infected with HCV, wherein the PBMCs have been activated and in which the HCV can replicate, together with a cell line, wherein the co-culturing enables infection of the cell line and replication of the HCV thereinto.

8. The system of claim 7, wherein said cell line is an immortalized cell line.

9. An assay for screening a test agent and selecting an agent which possesses anti-HCV activity, comprising:
a) growing a HCV infected cell according to the in vitro culture system of claim 1 and
b) assaying replication, translation, assembly infection or the like of HCV.

10. A method for identifying, from a library of compounds, a compound with anti-HCV activity, comprising:
a) providing a screening assay comprising a measurable biological activity of HCV;
b) contacting said screening assay with a test compound; and
c) detecting if said test compound inhibits the biological activity of HCV;
wherein a test compound which inhibits said biological activity is a compound with said inhibitory effect.

11. The method of claim 10, wherein the test compound with said therapeutic effect is further modified by combinatorial or medicinal chemistry to provide fuirther analogs of said test compound also having said therapeutic effect.

12. A compound having therapeutic effect on HCV, comprising:
a) providing a screening assay comprising a measurable biological activity of HCV;
b) contacting said screening assay with a test compound; and
c) detecting if said test compound inhibits the biological activity of HCV;
wherein a test compound which inhibits said biological activity is a compound with said inhibitory effect.

13. The compound of claim 12, wherein the compound with said therapeutic effect is further modified by combinatorial or medicinal chemistry to provide analogs of said compound also having said therapeutic effect.
